SEQUENCE LISTING (9 sequences)

SEQ ID NO: 1 - RNA, 20 nt, synthetic construct
  source 1..20: mol_type = other RNA
ctgaatctttggagtacctg

SEQ ID NO: 2 - RNA, 20 nt, synthetic construct
  source 1..20: mol_type = other RNA
tcacgtcatccagcagagaa

SEQ ID NO: 3 - RNA, 20 nt, synthetic construct
  source 1..20: mol_type = other RNA
cttgtcaaggctattggtca

SEQ ID NO: 4 - RNA, 20 nt, synthetic construct
  source 1..20: mol_type = other RNA
gctactctctctttctggcc

SEQ ID NO: 5 - RNA, 20 nt, synthetic construct
  source 1..20: mol_type = other RNA
gtcagggttctggatatctg

SEQ ID NO: 6 - RNA, 20 nt, synthetic construct
  source 1..20: mol_type = other RNA
gctggtacacggcagggtca

SEQ ID NO: 7 - RNA, 20 nt, synthetic construct
  source 1..20: mol_type = other RNA
agagtctctcagctggtaca

SEQ ID NO: 8 - RNA, 20 nt, synthetic construct
  source 1..20: mol_type = other RNA
tgtgctagacatgaggtcta

SEQ ID NO: 9 - RNA, 720 nt, synthetic construct
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

(19) United States
(12) Patent Application Publication
(10) Pub. No.: US 2026/0308636 A1
(43) Pub. Date: Oct. 8, 2026

(54) CELLULAR DELIVERY OF GENE EDITING COMPLEXES AND COMBINATIONS THEREOF

(71) Applicant: Portal Biotechnologies, Inc., Arlington, MA (US)

(72) Inventors: Rachel Eris CONOVER, Durham, NC (US); Jacquelyn Lee SIKORA HANSON, Sudbury, MA (US); Jonathan Brian GILBERT, Arlington, MA (US); Armon Reza SHAREI, Arlington, MA (US); Zhihui SONG, Philadelphia, PA (US); Sophia Madeleine HIRSCH, Boston, MA (US)

(21) Appl. No.: 19/484,507

(22) PCT Filed: May 14, 2024
(86) PCT No.: PCT/US2024/029330

§ 371 Date: Nov. 14, 2025

Related U.S. Application Data
(60) Provisional application No. 63/466,646, filed on May 15, 2023.

(51) Int. Cl. C12N 15/90

(52) U.S. Cl. CPC C12N 15/90

(57) ABSTRACT

In some aspects, provided herein is method for delivering to a cell a plurality of different gene editing complexes targeting a gene or a genetic motif wherein the method comprises subjecting a cell suspension comprising the cell to a mechanical deformation, such as material having pores therethrough, e.g., a filter. In other aspects, provided herein are systems, kits, and compositions useful for the methods taught herein.

### CROSS-REFERENCE TO RELATED APPLICATIONS

This application claims priority from U.S. provisional application 63/466,646, filed on May 15, 2023, the contents of which are incorporated by reference in their entirety.

### TECHNICAL FIELD

The present disclosure, in some aspects, is directed to methods for cellular delivery of gene editing complexes wherein the methods comprise subjecting a cell suspension to a mechanical deformation, such as material having pores therethrough, e.g., a filter. In other aspects, the disclosure is directed to systems, kits, and compositions useful for the methods taught herein.

### REFERENCE TO AN ELECTRONIC SEQUENCE LISTING

The content of the electronic sequence listing (285192000440seqlist.xml; Size: 9,656 bytes; and Date of Creation: May 14, 2024) is herein incorporated by reference in its entirety.

### BACKGROUND

Cellular engineering is a developing field, and delivery of cargos to a cell to accomplish such engineering is a significant challenge yet to be fully addressed. Techniques for delivering cargo to a cell are known, and include techniques involving electro-mechanical poration, chemical poration, and particle or viral-based delivery. However, there remains a need in the field for techniques for efficient delivery of more complex cargo or cargo mixtures that enable reasonable yields of the desired edited cells, wherein the cells maintain a robust viability for downstream uses. Moreover, such needs extend to the additional co-delivery of other cargos, such as nucleic acids.

### BRIEF SUMMARY

In some aspects, provided herein is a method of delivering to a cell a plurality of different gene editing complexes targeting a gene or genetic motif, the method comprising: subjecting a cell suspension to a mechanical deformation, the cell suspension comprising: (i) the cell, and (ii) the plurality of different gene editing complexes, or elements thereof, wherein the mechanical deformation is configured such that at least two of the plurality of different gene editing complexes enters the cell.

In some embodiments, the mechanical deformation is configured such that at least one copy of each of the plurality of different gene editing complexes enters the cell. In some embodiments, the at least one of the plurality of different gene editing complexes, or elements thereof, enters the cell as a complex. In some embodiments, the least one of the plurality of different gene editing complexes enters the cell as two or more elements thereof. In some embodiments, the plurality of different gene editing complexes comprises two or more different gene editing complexes. In some embodiments, the plurality of different gene editing complexes comprises 2-100 different gene editing complexes.

In some embodiments, the plurality of different gene editing complexes comprises a plurality of protein-nucleic acid complexes. In some embodiments, the plurality of different gene editing complexes comprises a plurality of ribonucleoprotein (RNP) complexes each comprising a protein element and a guide nucleic acid element. In some embodiments, the plurality of RNP complexes comprises two or more RNP complexes comprising the same protein element and different guide nucleic acid elements. In some embodiments, the method further comprises obtaining the plurality of RNP complexes, wherein the obtaining comprises loading the protein element with a guide nucleic acid element from a library of two or more different guide nucleic acid elements. In some embodiments, the plurality of different gene editing complexes comprises a Cas protein element. In some embodiments, the plurality of different gene editing complexes comprises a transposase.

In some embodiments, at least two of the plurality of different gene editing complexes target a respective editing region, and wherein the respective editing regions are non-overlapping. In some embodiments, at least two of the plurality of different gene editing complexes target a respective editing region, and wherein the respective editing regions are overlapping. In some embodiments, the plurality of different gene editing complexes is configured to edit one or both alleles of the gene. In some embodiments, the plurality of different gene editing complexes is configured to affect an insertion, deletion, modification, and/or substitution. In some embodiments, editing performed by the plurality of different gene editing complexes results in a knock-out of a gene. In some embodiments, editing performed by the plurality of different gene editing complexes results in a knock-in of a gene. In some embodiments, editing performed by the plurality of different gene editing complexes results in an epigenetic modification. In some embodiments, the plurality of different gene editing complexes edits B2M, TCRα subunit constant (TRAC), BCL11A, CCR5, or PD-1.

In some embodiments, the method further comprises delivering another gene editing complex to edit a second gene or genetic motif. In some embodiments, the gene or genetic motif is different than the second gene or genetic motif.

In some embodiments, the method further comprises co-delivering another cargo. In some embodiments, the other cargo is a nucleic acid or a polypeptide, or a complex comprising a combination of a nucleic acid and a polypeptide.

In some embodiments, the co-delivered nucleic acid is configured for genome integration. In some embodiments, the co-delivered nucleic acid is configured for protein expression. In some embodiments, the co-delivered nucleic acid is configured for knock-out or knock-down of a gene. In some embodiments, the co-delivered nucleic acid is a DNA, RNA, siRNA, miRNA, or oligonucleotide. In some embodiments, the co-delivered nucleic acid is configured for expression of any one of more of IL-2, IL-12, IL-15, IL-21, BCL-2, CXCR4, CCR7, TCF7, FOXO3, FOXp3, or a puromycin resistance gene.

In some embodiments, the mechanical deformation comprises passing the cell suspension, or a portion thereof, through a material having pores therethrough, a microfluidic device comprising a plurality of constrictions or confinements, or a microneedle or microneedle array, or subjecting the cell suspension, or a portion thereof, to high shear forces with or without an electric field.

In some embodiments, the mechanical deformation comprises passing the cell suspension, or a portion thereof, through the material having pores therethrough. In some embodiments, the material having pores therethrough is a filter. In some embodiments, the material having pores therethrough comprises a silicon-based material. In some embodiments, the material having pores therethrough comprises a polymer-based material. In some embodiments, the polymer-based material is a track-etched filter. In some embodiments, the material having pores therethrough comprises a metal-based material.

In some embodiments, the material having pores therethrough comprises pores having an average size of about 0.1 μm to about 300 μm. In some embodiments, the cell suspension is passed through the material having pores therethrough at about 1 psi to about 20 psi. In some embodiments, the cell suspension is passed through the material having pores therethrough at a cell speed of about 0.1 mm/second to about 5 m/second.

In some embodiments, the cell suspension has a volume of about 10 μL to about 5 L. In some embodiments, the cell suspension has a cell density of at least about 1,000 cells/mL. In some embodiments, the cell suspension comprises a buffer.

In some embodiments, the cell suspension comprises a plurality of the cells, and wherein, following the subjecting the cell suspension to the mechanical deformation, at least about 25% of the plurality of the cells receives at least one of the plurality of different gene editing complexes.

In some embodiments, the cell suspension comprises a plurality of the cells, and wherein, following the subjecting the cell suspension to the mechanical deformation, at least about 25% of the plurality of the cells are edited according to the design of at least one of the plurality of different gene editing complexes.

In some embodiments, the cell suspension comprises a plurality of the cells, and wherein, following the subjecting the cell suspension to the mechanical deformation, at least about 25% of the plurality of the cell are viable.

In some embodiments, the cell suspension comprises a plurality of the cells, and wherein, following the subjecting the cell suspension to the mechanical deformation, at least about 25% of the plurality of the cells that comprise at least one edit according to the design of at least one of the plurality of different gene editing complexes also contain the co-delivered nucleic acid.

In some embodiments, the cell is a somatic cell, or a stem cell, or a derivative thereof. In some embodiments, the cell is a PBMC, or a derivative thereof. In some embodiments, the cell is an immune cell. In some embodiments, the cell is a T cell, NK cell, monocyte, B cell, or dendritic cell. In some embodiments, the cell is a stem cell. In some embodiments, the cell is an induced pluripotent stem cell. In some embodiments, the cell is obtained, or derived, from an individual. In some embodiments, the individual is a human.

In other aspects, provided herein is a method of delivering to a cell a plurality of different ribonucleoprotein (RNP) complexes targeting a gene or a genetic motif, the method comprising: passing a cell suspension through a material having pores therethrough under a condition to mechanically deform the cell such that the plurality of different RNP complexes can enter the cell, wherein the cell suspension comprises: (i) the cell, and (ii) the plurality of different RNP complexes, or elements thereof.

In other aspects, provided herein is a method of delivering to a mixture of cells a plurality of different gene editing complexes targeting a gene or a genetic motif, wherein the mixture of cells comprises two or more different cell types, the method comprising: subjecting a cell suspension to a mechanical deformation, the cell suspension comprising: (i) the mixture of cells, and (ii) the plurality of different gene editing complexes, or elements thereof, wherein the mechanical deformation is configured such that at least two of the plurality of different gene editing complexes enter at least two different cell types of the mixture of cells. In some embodiments, the mixture of cells comprises different cell types. In some embodiments, the mixture of cells comprises PBMCs. In some embodiments, the mechanical deformation is configured such that at least one copy of each of the plurality of different gene editing complexes enters the portion of the mixture of cells comprising two or more cell types.

In other aspects, provided herein is a method of delivering to a cell a plurality of different gene editing complexes targeting a gene or a genetic motif, and another cargo, the method comprising: passing a cell suspension through a material having pores therethrough under a condition to mechanically deform the cell such that the plurality of different gene editing complexes and the other cargo can enter the cell, wherein the cell suspension comprises: (i) the cell, (ii) the plurality of different gene editing complexes, or elements thereof, and (iii) the other cargo.

In other aspects, provided herein is a system comprising: (a) a cell suspension comprising (i) a cell, and (ii) a plurality of different gene editing complexes; and (b) a device for subjecting the cell suspension to a mechanical deformation, wherein the device is configured in such that subjecting the cell suspension to the mechanical deformation allows the plurality of different gene editing complexes to enter the cell.

The disclosures of all publications, patents, patent applications and published patent applications referred to herein are hereby incorporated herein by reference in their entirety.

### BRIEF DESCRIPTION OF THE DRAWINGS

FIG. 1A shows a bar chart displaying results from a viability analysis of T cells mixed with RNP editing complexes and mechanically deformed by passage through low-porosity microsieve filters containing pore diameters of 4 μm or 5 μm at a pressure of 5 psi, 7 psi, or 10 psi.

FIGS. 1B and 1C shows the reduced surface expression efficiency of B2M expression in T cells following exposure to RNP genome editing complexes and mechanical deformation. FIG. 1B shows a bar chart displaying the percentage of B2M positive T cells for each condition. FIG. 1C shows a histogram displaying the population of T cells over a continuous plot of B2M surface expression of cells processed using the delivery methods described herein.

FIGS. 1D and 1E show Cascade Blue dextran delivery to T cells following mechanical deformation. FIG. 1D shows a bar chart displaying the percentage of Cascade Blue positive T cells for each condition. FIG. 1E shows a histogram displaying the population of T cells over a continuous plot of Cascade Blue fluorescence levels of cells processed using the delivery methods described herein.

FIG. 1F shows plots obtained from flow cytometry analysis of B2M surface expression and Cascade Blue fluorescence detected from cells processed using the delivery methods described herein.

FIG. 2A shows a bar chart displaying results from a viability analysis of T cells mixed with RNP editing complexes and mechanically deformed by passage through track-etched membranes containing a 5 μm pore diameter at a pressure of 5 psi, 7 psi, or 10 psi.

FIG. 2B shows a bar chart displaying results from a cell retention analysis following passage of cells through the track-etched filter. Cell retention values are relative to the “No Contact” control cells.

FIGS. 2C and 2D show the reduced surface expression efficiency of B2M expression in T cells following exposure to RNP genome editing complexes and mechanical deformation. FIG. 2C shows a bar chart displaying the percentage of B2M positive T cells for each condition. FIG. 2D shows a histogram displaying the population of T cells over a continuous plot of B2M surface expression of cells processed using the delivery methods described herein.

FIGS. 2E and 2F show Cascade Blue dextran delivery to T cells following mechanical deformation. FIG. 2E shows a bar chart displaying the percentage of Cascade Blue positive T cells for each condition. FIG. 2F shows a histogram displaying the population of T cells over a continuous plot of Cascade Blue fluorescence levels of cells processed using the delivery methods described herein.

FIG. 2G shows plots obtained from flow cytometry analysis of B2M surface expression and Cascade Blue fluorescence detected from cells processed using the delivery methods described herein.

FIG. 3A shows plots obtained from flow cytometry analysis of B2M surface expression and Cascade Blue fluorescence detected from cells processed using the delivery methods described herein.

FIG. 3B shows a bar chart displaying the percentage of T cells having edited the B2M locus following exposure to RNP genome editing complexes and mechanical deformation.

FIG. 3C shows histograms displaying the population of T cells over a continuous plot of Cascade Blue fluorescence levels (left) at day 0 following delivery and B2M expression levels (right) at day 2 following delivery of cells processed using the methods described herein.

FIG. 3D shows a bar chart displaying the percentage of live T cells having edited the B2M locus 2 days following exposure to RNP genome editing complexes and mechanical deformation.

FIG. 3E shows histograms displaying the population of T cells over a continuous plot of Cascade Blue fluorescence levels (left) at day 0 following delivery and B2M expression levels (right) at day 6 following delivery of cells processed using the methods described herein.

FIG. 3F shows a bar chart displaying the percentage of live T cells having edited the B2M locus 6 days following exposure to RNP genome editing complexes and mechanical deformation.

FIG. 3G shows plots obtained from flow cytometry analysis of B2M surface expression (y-axis) and GFP fluorescence (x-axis) detected from cells processed using the delivery methods described herein.

FIG. 3H shows a bar chart displaying the percentage of GFP positive, B2M negative, and Cas positive T cells following exposure to RNP genome editing complexes and mechanical deformation.

FIG. 3I shows a bar chart displaying results from a viability analysis of T cells mixed with two pools of RNP editing complexes and mechanically deformed.

FIG. 3J shows a bar chart displaying the percentage of B2M negative, and TRAC negative T cells following exposure to RNP genome editing complexes and mechanical deformation.

FIG. 3K shows plots obtained from flow cytometry analysis of B2M surface expression (y-axis) and TRAC expression (x-axis) detected from cells processed using the delivery methods described herein.

FIG. 4A shows a bar chart displaying results from a viability analysis of T cells following a sequential exposure to TRAC RNP editing complexes and mechanical deformation followed by a exposure to B2M RNP editing complexes and mRNA encoding GFP and mechanical deformation.

FIG. 4B shows a bar chart displaying the percentage of GFP positive, B2M negative, and TRAC negative T cells following sequential delivery of nucleic acid encoding GFP and RNP genome editing complexes targeting the B2M and TRAC loci.

FIG. 4C shows plots obtained from flow cytometry analysis of B2M surface expression (y-axis) and TRAC expression (x-axis) detected from cells processed using the sequential delivery methods described herein.

FIG. 4D shows a bar chart displaying the percentage of B2M KO, TRAC KO, or GFP expressing T cells following sequential delivery of TRAC sgRNA and dextran followed by delivery of a GFP expression construct, and either B2M sgRNA or B2M siRNA. For each experimental condition, the bars are ordered to show the percentage of B2M KO/KD cells, the percentage of TRAC KO cells, and the percentage of GFP expressing cells. KO=knockout, KD=knock down.

FIG. 4E shows histograms displaying the population of T cells over a continuous plot of Cascade Blue fluorescence levels (left) at day 0 following delivery, B2M expression levels (middle) at day 7, and GFP expression levels at day 7 following delivery of cells processed using the methods described herein.

FIG. 4F shows a bar chart displaying results from a viability analysis of T cells following a sequential exposure to B2M RNP editing complexes and mechanical deformation followed by a subsequent exposure to dextran and mechanical deformation.

FIG. 4G shows a bar chart displaying the percentage of Dextran positive and B2M negative T cells following sequential delivery of RNP genome editing complexes targeting the B2M locus and dextran.

FIG. 4H shows plots obtained from flow cytometry analysis of B2M surface expression (y-axis) and Cascade Blue Dextran fluorescence (x-axis) detected from cells processed using the sequential delivery methods described herein.

### DETAILED DESCRIPTION

Provided in the present application, in certain aspects, is a method of delivering to a cell a plurality of different gene editing complexes targeting a gene or genetic motif, the method comprising: subjecting a cell suspension to a mechanical deformation, the cell suspension comprising: (i) the cell, and, optionally, (ii) the plurality of different gene editing complexes, or elements thereof, wherein the mechanical deformation is configured such that at least two of the plurality of different gene editing complexes enters the cell. In some embodiments, the mechanical deformation comprises passing the cell suspension, or a portion thereof, through a material having pores therethrough, e.g., a filter. In some embodiments, the method further comprises co-delivering another cargo, such as a nucleic acid, to the cell. In some embodiments, the plurality of different gene editing complexes, or elements thereof, and/or the other cargo are placed in the presence of the cell when the cell contains pores following a mechanical deformation. In some embodiments, the cell suspension comprises the plurality of different gene editing complexes and/or the other cargo.

The present application is based, at least in part, on the inventors' unique perspectives and findings demonstrating that mechanical deformation of a cell can be used to efficiently deliver a plurality of different gene editing complexes targeting a single gene or genetic motif resulting in improved editing performance. For example, a pool of CRISPR-Cas editing systems comprising a single type of Cas protein, e.g., Cas9, loaded with different guide nucleic acid from a library targeting a single gene, e.g., B2M, can be readily delivered to a variety of clinically relevant cell types using the methods taught herein with the resulting engineering cells showing a desirable level of editing. Additionally, the inventors found that the methods taught herein are suitable for co-delivering another cargo, such as a polypeptide or nucleic acid such as RNA, sugar polymers, and other labeling agents, with a tight correlation of successful co-delivery with the plurality of different gene editing complexes. Said another way, it was found that a high percentage of cells exhibiting a desired edit from the plurality of different gene editing complexes also received delivery of the other cargo. As demonstrated herein, the taught methods also enable the delivery of multiple cargos with very different chemistries, e.g., a polypeptide (including those with net positive charge) versus a nucleic acid, which are generally negative charged species. This level of co-delivery is critical in cell engineering, e.g., engineering of cell therapies, as it is detrimental to the product performance and/or safety to produce a variable product (e.g., as would be produced if the cell received one cargo and not another). Moreover, the methods taught herein can be used to sequentially deliver cargos and obtain healthy cells containing said sequentially delivered cargos, or the effects thereof. It is well appreciated that sequential delivery with other techniques taught herein, e.g., electroporation, often results in poor cellular health and thus downstream uses of the processed cells are frustrated. As described herein, passing a cell through a material having pores therethrough to perform a mechanical deformation has been demonstrated for a number of material types, e.g., track-etched polymer-based filters and microsieves, and thus the delivery techniques provided herein are robust across different substrates. Moreover, such delivery techniques were demonstrated for clinically relevant cell types, including filter-based delivery of cargo to a human T cell, at clinical scale resulting in engineered cells suitable for clinical use. Furthermore, the methods taught herein do not significantly impact cell viability, enabling downstream uses of such engineered cells. These findings represent a significant advancement in the field of cellular delivery and engineering, and have use in advancing both the efficacy and safety of cell therapy production techniques.

Thus, provided herein, in some aspects, is a method of delivering to a cell a plurality of different gene editing complexes targeting a gene or genetic motif, the method comprising: subjecting a cell suspension to a mechanical deformation, the cell suspension comprising: (i) the cell, and (ii) the plurality of different gene editing complexes, or elements thereof, wherein the mechanical deformation is configured such that at least two of the plurality of different gene editing complexes enters the cell. In some embodiments, the mechanical deformation comprises passing the cell suspension, or a portion thereof, through a material having pores therethrough, e.g., a filter. In some embodiments, the method further comprises co-delivering another cargo, such as a nucleic acid, to the cell, such as wherein the cell suspension further comprises the other nucleic acid.

In other aspects, provided is a method of delivering to a cell a plurality of different ribonucleoprotein (RNP) complexes, e.g., CRISPR-Cas complexes, targeting a gene or a genetic motif, the method comprising: passing a cell suspension through a material having pores therethrough, e.g., a filter, under a condition to mechanically deform the cell such that a plurality of different RNP complexes can enter the cell, wherein the cell suspension comprises: (i) the cell, and (ii) the plurality of different RNP complexes, or elements thereof.

In other aspects, provided is a method of delivering to a mixture of cells a plurality of different gene editing complexes targeting a gene or a genetic motif, wherein the mixture of cells comprises two or more different cell types, the method comprising: subjecting a cell suspension to a mechanical deformation, the cell suspension comprising: (i) the mixture of cells, and (ii) the plurality of different gene editing complexes, or elements thereof, wherein the mechanical deformation is configured such that at least two of the plurality of different gene editing complexes enter at least two different cell types of the mixture of cells.

In other aspects, provided is a system comprising: (a) a cell suspension comprising (i) a cell, and (ii) a plurality of different gene editing complexes; and (b) a device for subjecting the cell suspension to a mechanical deformation, wherein the device is configured such that subjecting the cell suspension to the mechanical deformation allows the plurality of different gene editing complexes to enter the cell.

### I. Definitions

As used herein, the term “individual” refers to a mammal and includes, but is not limited to, human, bovine, horse, feline, canine, rodent, rat, mouse, dog, or primate (such as a non-human primate). In some embodiments, the individual is a human individual.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### II. Methods of Delivery

In certain aspects, provided herein are methods for cellular delivery of gene editing complexes wherein the methods comprise subjecting a cell suspension to a mechanical deformation, such as by passing the cell suspension through a material having pores therethrough, e.g., a filter.

In some embodiments, the method comprises subject a cell, such as in a cell suspension, to a mechanical deformation. In some embodiments, the method comprises passing a cell, such as in a cell solution, through a material having pores therethrough. In some embodiments, the cell is subjected to a mechanical deformation in the presence of a cargo, such as a plurality of gene editing complexes targeting a gene or genetic motif. For example, in some embodiments, the method is performed by subjecting a cell suspension comprising a cell and a cargo, such as a plurality of gene editing complexes targeting a gene or genetic motif, to a mechanical deformation, such as passing the cell suspension through a material comprising pores therethrough. In some embodiments, the cell is subjected to a mechanical deformation in the absence of a cargo, such as a plurality of gene editing complexes targeting a gene or genetic motif, wherein following deformation the cell is admixed with the cargo (such as providing the cargo during a period when pores still exist in a cell following a mechanical deformation). For example, in some embodiments, the method is performed by subjecting a cell suspension comprising a cell to a mechanical deformation, such as passing the cell suspension through a material comprising pores therethrough, followed by mixing the processed cell suspension with a cargo, such as a plurality of gene editing complexes targeting a gene or genetic motif.

The methods disclosed herein may comprise a variety of different combinations of the component features described herein. In some instances, such methods and component features are described in a modular fashion. The modular discussion of such component features does not limit the scope of the inventions provided herein and one of ordinary skill in the art will readily appreciate how certain features from the sections below can be combined to perform the cellular delivery methods provided herein.

### A. Mechanical Deformation Processes

In certain aspects, provided herein are mechanical deformation processes for use in the methods of delivery taught herein. Such mechanical deformation processes are suitable for deforming a cell such that pores exist in the cell for a short period of time (e.g., 30 seconds) after deformation allowing for cargo delivery to the inside of the cell, and after said period of time the pores close enclosing the delivered cargo within the cell.

In some embodiments, the mechanical deformation comprises passing a cell suspension, or a portion thereof, through a material having pores therethrough, e.g., a filter, a microfluidic device comprising a plurality of constrictions or confinements, or a microneedle or microneedle array, or subjecting the cell suspension, or a portion thereof, to high shear forces with or without the presence of an electric field. Examples of certain mechanical deformation processes, including associated devices, are found in U.S. Pat. No. 11,613,759 and Chakrabarty et al., Materials Today Bio, 13, 2022, the entire contents of each of which are incorporated by reference herein in their entirety.

As discussed in more detail below, in some embodiments, the method comprises passing a cell suspension, or a portion thereof including a cell, through a material having pores therethrough, e.g., a filter. In some embodiments, the cell is deformed by passing through a pore of the material having pores therethrough, and the processed cell can be collected in a filtrate after passing through the material.

### 1. Materials Having Pores Therethrough and Method Steps and Conditions Associated Therewith

In certain aspects, the methods of the present disclosure are directed to delivering cargos, such as a plurality of different gene editing complexes, into a cell by passing the cell through a material having pores therethrough, wherein the pores deform the cell, causing a perturbation of the cell and allowing delivery of the cargos, such as by having the cargos in the presence of the cell before, during, and/or after the cell is perturbed. In some embodiments, the material having pores therethrough is a filter.

The material having pores therethrough as disclosed herein can be made of any one of a number of materials, and combinations thereof, and take any one of a number of forms. In some embodiments, the material having pores therethrough is a filter (also referred to herein and in the art as a membrane). In some embodiments, the material having pores therethrough is a STERLITECH™ polycarbonate filter (PCT8013100). In some embodiments, the material having pores therethrough is a tangential flow filter. In some embodiments, the material having pores therethrough is a sponge or sponge-like matrix. In some embodiments, the material having pores therethrough is a matrix. In some embodiments, the material having pores therethrough is a microsieve. In some embodiments, the material having pores therethrough is not a net.

In some embodiments, the material having pores therethrough comprises a silicon-based material, such as silicon dioxide (also known as silica or SiO₂). In some embodiments, the material having pores therethrough comprises a silicon dioxide (also known as silica or SiO₂). In some embodiments, the material having pores therethrough comprises silicon nitride. In some embodiments, the material having pores therethrough comprises glass (also referred to as silicon oxide). In some embodiments, the material having pores therethrough comprises a polymer-based material, such as a polycarbonate. In some embodiments, the polymer-based material is a track-etched filter. In some embodiments, the material having pores therethrough comprises a metal-based material.

In some embodiments, the material having pores therethrough comprises a material having tortuous paths. In some embodiments, the material having tortuous paths comprises cellulose acetate. In some embodiments, the material having pores therethrough comprises a material selected from, without limitation, synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, graphite, and ceramic, or a mixture thereof.

The material having pores therethrough may be manufactured using any technique known in the art, including, without limitation, etching, track-etching, lithography, laser ablation, injection molding, stamping, micro-hole punching, polymeric-sponge, direct foaming, extrusion, and hot embossing. Etching involves the process of using a chemical, such as a strong acid, to cut into a material, such as a metal, in a desired pattern. Track-etched materials are formed by bombarding a solid film with particles that form tracks of damaged material through the film. The film is then subjected to a chemical agent that selectively etches the damaged tracks to create perforations through the film. The cross-sectional widths of the pores can be controlled by the incubation time of the etchant on the film. Lithography can include microlithography, nanolithography, and x-ray lithography. In lithography, a lithographic apparatus applies a desired pattern onto a target portion of the substrate. For example, in x-ray lithography, x-rays are used to transfer a pattern from a mask to a light-sensitive chemical photoresist on the desired substrate. In photolithography, light is used to transfer the desired pattern from a photomask to a light-sensitive photoresist on the substrate. Subsequent chemical application is used to engrave the pattern into the substrate material beneath the photoresist. Laser ablation is the process of removing material from a solid surface by irradiating with a laser beam, while injection molding involves injecting a material into a desired mold, where it then cools and hardens into the desired structure. Stamping and micro-hole punching methods utilize tools to cut or imprint desired forms into the substrate material. To produce ceramic filter membranes, the polymeric-sponge method involves saturating a polymeric sponge with a ceramic slurry, which is then burned out to leave a porous ceramic. In the direct foaming method, a chemical mixture containing the desired ceramic component and organic materials is treated to evolve a gas. Bubbles are then produced in the material, causing it to foam. The resulting porous ceramic material is then dried and fired. To produce a honeycomb or cellular structure, a plastic-forming method called extrusion is used, where a mixture of ceramic powder plus additives is forced through a shaped die. Hot embossing involves the stamping of a pattern into a polymer softened by raising the temperature of the polymer just above its glass transition temperature.

In some embodiments, the material having pores therethrough is a nanostructure membrane. In some embodiments, phase change is used to produce nano structured membranes. Phase change methods include, without limitation, precipitation from the vapor phase, dry-wet phase inversion, and thermally induced phase separation. In the precipitation from the vapor phase method, a cast polymer solution composed of polymer and solvent is introduced into a nonsolvent vapor environment saturated with solvent vapor. The saturated solvent vapor suppresses the evaporation of solvent from the film. The nonsolvent molecules subsequently diffuse into the film, leading to polymer setting. In the dry-wet phase inversion method, a polymer solution composed of polymer and solvent is prepared. The solution is cast on a suitable surface and after partial evaporation of the solvent, the cast film is immersed in a gelatin. Then, the nonsolvent diffuses into the polymer solution film through the thin solid layer while the solvent diffuses out, creating a porous membrane. In the thermally induced phase separation method, a polymer is mixed with a solvent at a high temperature and the polymer solution is cast into a film. When the solution is cooled, it enters into an immiscible area due to the loss of solvency.

In some embodiments, the material having pores therethrough comprise a block copolymer. Block copolymers are composed of two or more blocks of different polymerized monomers linked by covalent bonds. Block copolymer components can microphase separate to form periodic nanostructures. In this process, due to incompatibility between the blocks, block copopolymers undergo phase separation, creating nanometer-sized structures.

In some embodiments, the material having pores therethrough comprises a microsieve. In some examples, microsieves are used in cell separation, CTC isolation or droplet emulsification techniques. In some embodiments, pores of a microsieve are generated by photolithography on a ceramic substrate with a silicon support. In some embodiments, the microsieve is made of polycarbonate. Microsieves are available from, e.g., Aquamarijn. In some embodiments, the microsieve has a pore size ranging from about 2 μm to about 10 μm, such as about 4 μm to about 10 μm. In some embodiments, the microsieve has a porosity of greater than about any of 5%, 8%, 10%, 15%, 20%, 25%, 30%, 35%, 40%, 45%, or 50%.

The material having pores therethrough may be characterized by a surface, such as a top surface or a surface across a cross-section of a material, upon which a cell suspension first contacts the material when the cell suspension is passed through the material having pores therethrough. In some embodiments, the surface is substantially perpendicular to the general direction of flow a cell suspension will take through a material having pores therethrough when said cell suspension is passed through the material. The 2-dimensional shape of the surface can be any shape, such as, without limitation, circular, elliptical, round, square, star-shaped, triangular, polygonal, pentagonal, hexagonal, heptagonal, or octagonal. In some embodiments, the surface is round in shape. In some embodiments, the surface of the material having pores therethrough has a largest cross-sectional width of about 1 mm to about 3 m, such as any of about 1 mm to about 750 mm, about 1 mm to about 500 mm, about 1 mm to about 250 mm, about 1 mm to about 100 mm, about 1 mm to about 50 mm, about 1 mm to about 25 mm, about 1 mm to about 10 mm, about 1 mm to about 5 mm, or about 1 mm to about 2.5 mm.

The surface of a material having pores therethrough may be substantially flat (such as having a surface variance as measured by two parallel planes of 1 mm or less). In some embodiments, the surface of a material having pores therethrough, or a portion thereof, has a designed topography, including having a surface variance as measured by two parallel planes of more than 1 mm.

The material having pores therethrough may be characterized by a thickness. In some embodiments, the thickness of a material is substantially parallel to the general direction of flow a cell suspension will take through a material having pores therethrough when said cell suspension is passed through the material. In some embodiments, the thickness of a material having pores therethrough is uniform. In some embodiments, the thickness of a material having pores therethrough is variable. For example, in some embodiments, portions of a material having pores therethrough are thicker or thinner than other portions of the material having pores therethrough. In some embodiments, the surface thickness varies by about 1-90% as compared to the smallest thickness. In some embodiments, the material having pores therethrough has a thickness of about 0.01 μm to about 20 mm, such as any of about 0.01 μm to about 5 mm, about 0.01 μm to about 2.5 mm, about 0.01 μm to about 1 mm, about 0.01 μm to about 750 μm, about 0.01 μm to about 500 μm, about 0.01 μm to about 250 μm, about 0.01 μm to about 100 μm, about 0.01 μm to about 90 μm, about 0.01 μm to about 80 μm, about 0.01 μm to about 70 μm, about 0.01 μm to about 60 μm, about 0.01 μm to about 50 μm, about 0.01 μm to about 40 μm, about 0.01 μm to about 30 μm, about 0.01 μm to about 20 μm, 1 μm to about 20 μm, about 0.01 μm to about 10 μm, about 0.01 μm to about 5 μm, about 0.01 μm to about 1 μm, about 0.01 μm to about 0.5 μm, about 0.01 μm to about 0.1 μm, about 0.01 μm to about 0.05 μm, about 0.05 μm to about 5 mm, about 0.1 μm to about 5 mm, about 0.5 μm to about 5 mm, about 1 μm to about 5 mm, about 5 μm to about 5 mm, about 10 μm to about 5 mm, about 20 am to about 5 mm, about 30 μm to about 5 mm, about 40 μm to about 5 mm, about 50 μm to about 5 mm, about 60 μm to about 5 mm, about 70 μm to about 5 mm, about 80 μm to about 5 mm, about 90 μm to about 5 mm, about 100 μm to about 5 mm, about 250 μm to about 5 mm, about 500 μm to about 5 mm, about 750 μm to about 5 mm, about 1 mm to about 5 mm, or about 2.5 mm to about 5 mm. In some embodiments, the material having pores therethrough has a thickness of about 1 μm to about 20 μm, such as about any of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm.

In some embodiments, the material having pores therethrough comprises a surface, such as a top surface or a cross-sectional surface, having a largest cross-sectional width of about 1 mm to about 3 m, including any sub-ranges (such as disclosed herein) and a thickness of about 0.01 μm to about 20 mm, including any sub-ranges (such as disclosed herein).

The material having pores therethrough can be formed in a variety of different shapes, including 3-dimensional shapes. In some embodiments, the 3-dimensional shape is cylindrical, conical, or cuboidal.

The material having pores therethrough can be characterized by the size(s) of the pores. In some embodiments, the pore size described herein reflects the smallest cross-sectional width across a pore. In some embodiments, the pore size described herein reflects the average cross-sectional width across a pore, such as evaluated for a single pore across the thickness of a material having pores therethrough and/or for a population of pores. In some embodiments, the pore size reflects an average of a population of pores of a material having pores therethrough. In some embodiments, the pores are characterized by a cross-sectional shape of the pores or an aspect (such as observed for a thickness of the material) thereof. In some embodiments, the pores have a cross-sectional shape that is circular or roughly circular. In some embodiments, the pores have a cross-sectional shape that is polygonal (e.g., triangular, square, rectangular, pentagonal, or hexagonal). In some embodiments, the cross-sectional shape of a pore is circular, elliptical, square, rectangular, star-shaped, triangular, polygonal, pentagonal, hexagonal, heptagonal, or octagonal.

In some embodiments, the material having pores therethrough comprises pores having a size, such as a cross-sectional width, selected based on the cell being passed therethrough. For example, in some embodiments, the smallest cross-sectional width across a pore is about 99% or less, such as about any of 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, or 20% or less, the average size of a cell (such as measured by the diameter or cross-sectional distance of the cell). In some embodiments, the cell has a cross-sectional width (e.g., diameter) in the cell suspension of between about 5-15 μm, e.g., dendritic cells are 7-8 μm in diameter. In another example, the cross-sectional width (e.g., diameter) of a human ovum is about 120 μm. In some embodiments, the cross-sectional width of pores of a material described herein is about 0.01 μm to about 300 μm, such as about 0.01 μm to about 250 μm, about 0.01 μm to about 200 μm, about 0.01 μm to about 150 μm, about 0.01 μm to about 100 μm, about 0.01 μm to about 50 μm, about 0.01 μm to about 25 μm, about 25 μm to about 300 μm, about 50 μm to about 300 μm, about 100 μm to about 300 μm, about 150 μm to about 300 μm, about 200 μm to about 300 μm, or about 250 μm to about 300 μm. In some embodiments, the pore cross-sectional width is about any of 10 μm or less, 9.5 μm or less, 9.0 μm or less, 8.5 μm or less, 8.0 μm or less, 7.5 μm or less, 7.0 μm or less, 6.5 μm or less, 6.0 μm or less, 5.5 μm or less, 5.0 μm or less, 4.5 μm or less, 4.0 μm or less, 3.5 μm or less, 3.0 μm or less, 2.5 μm or less, 2.0 μm or less, 1.5 μm or less, 1.0 μm or less, or 0.5 μm or less. In some embodiments, the pores are heterogeneous in cross-sectional width. In some embodiments, the pores are substantially homogenous (such as within about 20% or less variance) in cross-sectional width.

In some embodiments, the material having pores therethrough comprises pores having an average size of about 0.1 μm to about 300 μm, such as about any of 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm. In some embodiments, the pore has a uniform cross-sectional dimension along the length of the pore passage, e.g., through the thickness of the material having pores therethrough. In some embodiments, the pore has a variable cross-sectional dimension along the length of the pore passage, e.g., increasing or decreasing.

In some embodiments, the entrances and exits of a pore through a material having pores therethrough may have a variety of angles. In some embodiments, the pore angle can be selected to minimize clogging of the pore while cells are passing through. For example, the angle of the entrance or exit portion can be between about 0 and about 90 degrees. In some embodiments, the pores have identical entrance and exit angles. In some embodiments, the pores have different entrance and exit angles. In some embodiments, the pore edge is smooth, e.g., rounded or curved. A smooth pore edge has a continuous, flat, and even surface without bumps, ridges, or uneven parts. In some embodiments, the pore edge is sharp. A sharp pore edge has a thin edge that is pointed or at an acute angle. In some embodiments, the pore passage is straight. A straight pore passage does not contain curves, bends, angles, or other irregularities. In some embodiments, the pore passage is curved. A curved pore passage is bent or deviates from a straight line. In some embodiments, the pore passage has multiple curves, e.g., about 2 or more, 3 or more, 4 or more, 5 or more, 6 or more, 7 or more, 8 or more, 9 or more, or 10 or more. In some embodiments, the pore has a fluted entrance and exit shape. In some embodiments, the pore shape is homogenous among other pores within a given material or a portion thereof. In some embodiments, the pore shape is heterogeneous (i.e., mixed or varied) among pores within a given material or a portion thereof.

The material having pores therethrough described herein may have a range of total pore numbers and/or area sizes having said pores. In some embodiments, the pores encompass about 10-80% of the total surface area of a material having pores therethrough. In some embodiments, the material having pores therethrough comprises about 1.0×10³ to about 1.0×10³⁰ pores, including, e.g., at least about any of 1.0×10³ pores, 1.0×10⁴ pores, 1.0×10⁵ pores, 1.0×10⁶ pores, 1.0×10⁷ pores, 1.0×10⁸ pores, 1.0×10⁹ pores, 1.0×10¹⁰ pores, 1.0×10¹¹ pores, 1.0×10¹² pores, 1.0×10¹³ pores, 1.0×10¹⁴ pores, 1.0×10¹⁵ pores, 1.0×10¹⁶ pores, 1.0×10¹⁷ pores, 1.0×10¹⁸ pores, 1.0×10¹⁹ pores, 1.0×10²⁰ pores, 1.0×10²¹ pores, 1.0×10²² pores, 1.0×10²³ pores, 1.0×10²⁴ pores, 1.0×10²⁵ pores, 1.0×10²⁶ pores, 1.0×10²⁷ pores, 1.0×10²⁸ pores, 1.0×10²⁹ pores, or 1.0×10³⁰ pores. In some embodiments, the material having pores therethrough comprises about 10 to about 1.0×10¹⁵ pores per mm².

The pores can be distributed in numerous ways within a given material having pores therethrough. In some embodiments, the pores are distributed in grid as viewed from a surface of the material. In one such example, the pores are distributed side-by-side as viewed from a surface of the material, including having substantially equal distancing from one another. In some embodiments, the pore distribution is ordered or homogeneous as viewed from a surface of the material. In some embodiments, the pores are distributed in a regular, systematic pattern as viewed from a surface of the material. In some embodiments, the pore distribution is random or heterogeneous as viewed from a surface of the material. In some embodiments, the pores are distributed in an irregular, disordered pattern or are different distances apart as viewed from a surface of the material. In some embodiments, the pores are arranged in an irregular pattern as viewed from a surface of the material. In some embodiments, the pores are heterogeneous in size, such as pore size. In some embodiments, the pores are heterogeneous in shape, such as cross-sectional shape.

In some embodiments, the material having pores therethrough comprises more than one separate piece, such as a first material piece and a second material piece. In some embodiments, the pieces of material constituting the material having pores therethrough are arranged in series. In some embodiments, the pieces of a material having pores therethrough are arranged such that a cell solution travels through each piece in series. In some embodiments, at least two of the pieces of a material constituting the material having pores therethrough are the same. In some embodiments, at least two of the pieces of a material constituting the material having pores therethrough are different.

In some embodiments, the surface of a material having pores therethrough comprises a coating. Such coating can be selected from any material known in the art, including, without limitation, Teflon, an adhesive coating, surfactants, anticoagulants such as heparin, EDTA, citrate, and oxalate, proteins, adhesion molecules, antibodies, factors that modulate cellular function, nucleic acids, lipids, carbohydrates, complexes such as lipid-carbohydrate complexes, or transmembrane proteins. In some embodiments, the coating comprises, including is, polyvinylpyrrolidone. In some embodiments, the coating is covalently attached to a surface. In some embodiments, the coating is non-covalently attached to a surface. In some embodiments, the coating is released, or removed, as the cells pass through the pores.

In some embodiments, the material having pores therethrough comprises a modified mechanical or chemical property, e.g., exposed aspects thereof (such as a surface and/or pore) are modified to have a desired property such as hydrophilicity, hydrophobicity, charge (positive, negative, neutral, or zwitter ionic), or smoothness.

In some embodiments, the material having pores therethrough is configured with a directionality, e.g., configured such that a cell suspension is intended to enter and exit specific portions of a material having pores therethrough.

As described herein, the delivery methods comprise subjecting a cell suspension to a mechanical deformation. Such mechanical deformation may be performed under certain conditions, such as based on pressure and/or flow rate of the cell suspension.

In some embodiments, the cell suspension is passed through a material having pores therethrough at about 1 psi (pounds per square inch) to about 100 psi, such as any of about 1 psi to about 10 psi, about 1 psi to about 20 psi, about 5 psi to about 12 psi, or about (5 psi to about 10 psi). In some embodiments, the cell suspension is passed through a material having pores therethrough at about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, 21 psi, 22 psi, 23 psi, 24 psi, 25 psi, 26 psi, 27 psi, 28 psi, 29 psi, 30 psi, 31 psi, 32 psi, 33 psi, 34 psi, 35 psi, 36 psi, 37 psi, 38 psi, 39 psi, 40 psi, 41 psi, 42 psi, 43 psi, 44 psi, 45 psi, 46 psi, 47 psi, 48 psi, 49 psi, 50 psi, 51 psi, 52 psi, 53 psi, 54 psi, 55 psi, 56 psi, 57 psi, 58 psi, 59 psi, 60 psi, 61 psi, 62 psi, 63 psi, 64 psi, 65 psi, 66 psi, 67 psi, 68 psi, 69 psi, 70 psi, 71 psi, 72 psi, 73 psi, 74 psi, 75 psi, 76 psi, 77 psi, 78 psi, 79 psi, 80 psi, 81 psi, 82 psi, 83 psi, 84 psi, 85 psi, 86 psi, 87 psi, 88 psi, 89 psi, 90 psi, 91 psi, 92 psi, 93 psi, 94 psi, 95 psi, 96 psi, 97 psi, 98 psi, 99 psi, or 100 psi. In some embodiments, the pressure is as measured upstream of a material having pores therethrough, e.g., in a cell suspension prior to passing through the material having pores therethrough. In some embodiments, the pressure is held within +/− about 20% variance of a desired pressure during the period of time that a cell suspension is passed through a material having pores therethrough. In some embodiments, two or more pressures are used to pass a cell suspension through a material having pores therethrough. In some embodiments, the pressure for passing a cell suspension through a material having pores therethrough is a positive pressure, such as created by a pump or syringe pushing a cell suspension through a material having pores therethrough. In some embodiments, the pressure for passing a cell suspension through a material having pores therethrough is a negative pressure, such as created by a vacuum source pulling a cell suspension through a material having pores therethrough. Techniques for passing a cell suspension through a material having pores therethrough are understood by one of skill in the art and include, e.g., a peristaltic pump, a centrifugal pump, a syringe pump, a piston pump, rotary vane pump, diaphragm pump, turbomolecular pump, or a scroll pump, or any combination or hybrid thereof.

In some embodiments, the cell suspension is passed through the material having pores therethrough at a cell speed of about 0.1 mm/second to about 5 m/second, such as about any of 0.1 mm/second to about 1 mm/second, about 1 mm/second to about 1 cm/second, about 1 cm/second to about 10 cm/second, about 10 cm/second to about 25 cm/second, about 25 cm/second to about 50 cm/second or about 50 cm/second to about 100 cm/second, about 100 cm/second to about 500 cm/second, about 500 cm/second to about 1 m/second, or about 1 m/second to about 5 m/second. In some embodiments, the flow rate of a cell suspension through a material having pores therethrough is between about 0.001 mL/cm²/second to about 100 L/cm²/second, such as any of about 0.001 mL/cm²/second to about 75 L/cm²/second, about 0.001 mL/cm²/second to about 50 L/cm²/second, about 0.001 mL/cm²/second to about 25 L/cm²/second, about 0.001 mL/cm²/second to about 10 L/cm²/second, about 0.001 mL/cm²/second to about 7.5 L/cm²/second, about 0.001 mL/cm²/second to about 5.0 L/cm²/second, about 0.001 mL/cm²/second to about 2.5 L/cm²/second, about 0.001 mL/cm²/second to about 1 L/cm²/second, about 0.001 mL/cm²/second to about 0.1 L/cm²/second, about 0.001 mL/cm²/second to about 75 mL/cm²/second, about 0.001 mL/cm²/second to about 50 mL/cm²/second, about 0.001 mL/cm²/second to about 25 mL/cm²/second, about 0.001 mL/cm²/second to about 10 mL/cm²/second, about 0.001 mL/cm²/second to about 1 mL/cm²/second, about 0.001 mL/cm²/second to about 0.1 mL/cm²/second, about 0.001 mL/cm²/second to about 0.01 mL/cm²/second, about 0.001 mL/cm²/second to about 100 L/cm²/second, about 0.01 mL/cm²/second to about 100 L/cm²/second, about 0.1 mL/cm²/second to about 100 L/cm²/second, about 1 mL/cm²/second to about 100 L/cm²/second, about 10 mL/cm²/second to about 100 L/cm²/second, about 50 mL/cm²/second to about 100 L/cm²/second, about 0.1 L/cm²/second to about 100 L/cm²/second, about 0.5 L/cm²/second to about 100 L/cm²/second, about 1 L/cm²/second to about 100 L/cm²/second, about 2.5 L/cm²/second to about 100 L/cm²/second, about 5 L/cm²/second to about 100 L/cm²/second, about 7.5 L/cm²/second to about 100 L/cm²/second, about 10 L/cm²/second to about 100 L/cm²/second, about 25 L/cm²/second to about 100 L/cm²/second, about 50 L/cm²/second to about 100 L/cm²/second, or about 75 L/cm²/second to about 100 L/cm²/second.

### B. Gene Editing Complexes

The methods provided herein are configured for delivering to a cell a plurality of different gene editing complexes targeting a gene or a genetic motif. As contemplated herein, in some embodiments, targeting a gene encompasses editing (such as by deletion, modification, substitution, and/or addition) any one of more of the following: a coding portion of a gene, a non-coding portion of a gene, or a genetic element that controls expression of a gene, such as a promoter or an epigenetic feature. As contemplated herein, in some embodiments, targeting a genetic motif encompasses editing (such as by deletion, modification, substitution, and/or addition) genomic DNA based on a nucleic acid sequence recognized by a gene editing complex, wherein said genetic motif may be found in one or more genes or a non-coding portion of the genome. In some embodiments, editing a gene or a genetic motif comprises gene editing complexes that specifically bind DNA, such as to silence or temporarily block expression or function or provide activation, e.g., CRISPR-based activation and inhibition/interference (e.g., CRISPRa and CRISPRi). As described herein, such methods also enable the co-delivery of additional cargo, including, e.g., another gene editing complex targeting a different gene or genetic motif.

In some embodiments, the plurality of different gene editing complexes comprises 2 or more, such as any of 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more, different gene editing complexes. In some embodiments, the plurality of different gene editing complexes comprises 2-100, such as any of 2-50, 2-25, 2-20, 2-10, 2-5, or 3-4, different gene editing complexes. In some embodiments, the plurality of different gene editing complexes is 2, 3, 4, 5, 6, 7, 8, 9, 10, 11, 12, 13, 14, 15, 16, 17, 18, 19, or 20 different gene editing complexes.

The types of gene editing complexes contemplated herein are known in the art, e.g., CRISPR-Cas systems as described in Makarova & Koonin, Methods Mol Biol, 1311, 2015, and transposase systems as described in Sandoval-Villegas et al., Int J Mol Sci, 22, 2021, the entire contents of each of which are incorporated by reference herein in their entirety

Furthermore, editing abilities of such gene editing complexes are well appreciated in the art. For example, in some embodiments, at least one, including all, of a plurality of gene editing complexes is configured to edit one or both alleles of a gene. In some embodiments, at least one, including all, of a plurality of different gene editing complexes is configured to affect an insertion, deletion, modification, and/or substitution, such as a base edit. In some embodiments, editing performed by at least one, including all, of a plurality of different gene editing complexes results in a knock-out of a gene. In some embodiments, editing performed by at least one, including all, of a plurality of different gene editing complexes results in a knock-in of a gene. In some embodiments, editing performed by at least one, including all, of a plurality of different gene editing complexes results in a base edit, e.g., a base substitution. In some embodiments, editing performed by at least one, including all, of a plurality of different gene editing complexes results in an epigenetic modification. In some embodiments, at least one of a plurality of different gene editing complexes is configured to provide the desired change to a cell, e.g., a single gene editing complex knocks out a desired gene. In some embodiments, two or more of a plurality of different gene editing complexes are configured to provide the desired change to a cell. In some embodiments, two or more of a plurality of different gene editing complexes are configured to redundantly provide the desired change to a cell, e.g., two or more different gene editing complexes independently configured to knock out a gene.

In some embodiments, gene editing complexes provide an edit within an editing region, such as a stretch of greater than one nucleic acid. In such embodiments, at least two of the plurality of different gene editing complexes perform edits within a respective editing region, and wherein the respective editing regions are non-overlapping. In such embodiments, at least two of the plurality of different gene editing complexes perform edits within a respective editing region, and wherein the respective editing regions are overlapping.

In some embodiments, the process for mechanical deformation (such as described elsewhere in the application) is configured such that at least one of a plurality of gene editing complexes, or elements thereof, enters the cell as a complex. In some embodiments, the least one of the plurality of different gene editing complexes enters the cell as two or more elements thereof, e.g., a Cas protein not loaded with a guide nucleic acid wherein the gene editing complex assembles itself in the cell after loading. In some embodiments, the process for mechanical deformation (such as described elsewhere in the application) is configured such that at least about 50%, such as at least about any of 55%, 60%, 65%, 70%, 75%, 80%, 85%, 90% or 95%, of different gene loading complexes, or elements thereof, provided for delivery to a cell enter the cell. In some embodiments, the process for mechanical deformation (such as described elsewhere in the application) is configured such that all different gene loading complexes, or elements thereof, provided for delivery to a cell enter the cell, e.g., 4 different gene editing complexes targeting a gene or a gene motif admixed in a cell suspension and then passed through a material having pores therethrough enter the cell. In some embodiments, the process for mechanical deformation (such as described elsewhere in the application) is configured such that at least one copy, including between 2 to 1,000,000 copies, such as any of 2 to 100,000 copies, including at least about any of 10 copies, 25 copies, 50 copies, 100 copies, 500 copies, 1,000 copies, 2,500 copies, 5,000 copies, or 10,000 copies, of at least two more, including each, of a plurality of different gene editing complexes enters a cell.

In some embodiments, the plurality of different gene editing complexes comprises a plurality of protein-nucleic acid complexes. In some embodiments, the plurality of different gene editing complexes comprises a plurality of ribonucleoprotein (RNP) complexes each comprising a protein element and a guide nucleic acid element. In some embodiments, the plurality of RNP complexes comprises two or more RNP complexes comprising the same protein element and different guide nucleic acid elements. In some embodiments, the plurality of RNP complexes comprises two or more RNP complexes comprising different protein elements and the same guide nucleic acid element. In some embodiments, the plurality of RNP complexes comprises two or more RNP complexes comprising different protein elements and different guide nucleic acid elements.

In some embodiments, the method further comprises obtaining a plurality of RNP complexes. In some embodiments, the obtaining comprises loading a protein element with a guide nucleic acid element from a library of two or more different guide nucleic acid elements. In some embodiments, the method further comprises obtaining one or more compositions comprising, including separately comprising, elements of a plurality of RNP complexes, such as a composition comprising one or more protein elements and one or more guide nucleic acids of a plurality of RNP complexes, or a first composition comprising one or more protein elements of a plurality of RNP complexes and a second composition comprising one or more guide nucleic acids of the plurality of RNP complexes.

In some embodiments, the plurality of different gene editing complexes comprises a Cas protein element. CRISPR-Cas systems are well known in the art, e.g., as described in Makarova & Koonin, Methods Mol Biol, 1311, 2015, and generally comprise a protein element, namely, Cas, and a nucleic acid element, namely, a guide nucleic acid for targeting a specific genomic sequence. In some embodiments, the CRISPR-Cas system is a Type I system, including any of subtypes I-A, I-B, I-C, I-C variant, I-D, I-E, I-F, I-F variant I, or I-F variant II. In some embodiments, the CRISPR-Cas system is a Type II system, including any of subtypes II-A, II-B, or II-C. In some embodiments, the CRISPR-Cas system is a Type III system, including any of subtypes III-A, III-B, III-B variant. In some embodiments, the CRISPR-Cas system is a Type IV system or Type IV system variant. In some embodiments, the protein element of a gene editing complex is selected from the group consisting of Cas9, Cas3, Cas10, Cas8, Cas5, Cas12, or subtypes/variants thereof. In some embodiments, the gene editing complex comprises Cas9. In some embodiments, the gene editing complex comprises Cas12a. In some embodiments, the gene editing complex comprises dCas9 (dead Cas9).

In some embodiments, the plurality of different gene editing complexes comprises a moveable genetic element (MGE). In some embodiments, the plurality of different gene editing complexes comprises a transposase, such as described in Sandoval-Villegas et al., Int J Mol Sci, 22, 2021. In some embodiments, the plurality of different gene editing complexes comprises a PRIME editor, such as described in Scholefield & Harrison, Gene Therapy, 28, 2021, the entire contents of which are incorporated by reference herein in their entirety. In some embodiments, the plurality of different gene editing complexes comprises a PASTE editing system, such as described in Yarnall et al., Nature Biotechnology, 41, 2023, the entire contents of which are incorporated by reference herein in their entirety.

In some embodiments, the gene or gene motif targeted by a gene editing complex is B2M, TCRα subunit constant (TRAC), BCL11A, CCR5, or PD-1.

### C. Co-Delivery

The methods provided herein further enable the co-delivery of additional cargo, including, e.g., a polypeptide, a nucleic acid molecule, a gene editing complex (such as targeting another gene or genetic motif), or elements thereof, or a chemical compound such as a small molecule (e.g., 2 kDa or less in size). As described herein, co-delivery comprises processing a cell such that a plurality of different gene editing complexes enters the cell and another cargo (such as at least one other cargo) enters the cell. In some embodiments, the co-delivery is performed in a unified step (e.g., the plurality of different gene editing complexes and the other cargo are simultaneously introduced to the cell, e.g., via a single mechanical deformation process), such as using a cell suspension comprising the plurality of different gene editing complexes and the other cargo. In some embodiments, the co-delivery is performed by sequentially or concurrently exposing a cell to a plurality of gene editing complexes and another cargo, e.g., such as passing a cell suspension comprising the plurality of different gene editing complexes through a material having pores therethrough and then subsequently adding the other cargo while the cells still contain pores from the mechanical deformation. In some embodiments, provided herein are methods comprising two or more steps of subjecting a cell suspension to a mechanical deformation, e.g., subjected a cell suspension to a mechanical deformation to deliver a plurality of different gene editing complexes, or elements thereof, wherein the mechanical deformation is configured such that at least two of the plurality of different gene editing complexes enters the cell, and subjecting the cell suspension to a mechanical deformation to deliver one or more other cargos, including one or more gene editing complexes, a nucleic acid, such as RNA, e.g., mRNA or siRNA, a sugar polymer, or other labeling agent. In some embodiments, the methods allow for the passage of time between mechanical deformation steps, such as to expand cells following a delivery step. For example, in some embodiments, the methods comprise at least about 1 day, such as at least about any of 2, 3, 4, 5, 6, 7, 8, 9, 10, 12, 13, 14, 21, 28, 35, or 42 days between a first mechanical deformation step and a second mechanical deformation step. One of ordinary skill in the art will readily recognize that the cell suspension may change between the mechanical deformation steps, such as accomplished by changing cellular media, buffer exchange, or cell replication and/or death and/or change (e.g., differentiation and/or activation), however use of this identifier in subsequent steps is to convey the lineage between multiple rounds of mechanical deformation. In some embodiments, the methods provided herein comprise subjecting a cell suspension to a mechanical deformation to deliver a plurality of different gene editing complexes, or elements thereof, and subjecting the cell suspension to a mechanical deformation to deliver a nucleic acid, such as RNA, e.g., mRNA. In some embodiments, the methods provided herein comprise subjecting a cell suspension to a mechanical deformation to deliver a plurality of different gene editing complexes, or elements thereof, and subjecting the cell suspension to a mechanical deformation to deliver a nucleic acid, such as RNA, e.g., mRNA. In some embodiments, the co-delivered cargo is a polypeptide, a nucleic acid (such as RNA, e.g., mRNA, or DNA), or a complex, such as a complex comprising a polypeptide and a nucleic acid. In some embodiments, the polypeptide is a peptide. In some embodiments, the polypeptide is a protein.

In some embodiments, the method further comprises co-delivering a nucleic acid. In some embodiments, the co-delivered nucleic acid is configured for genome integration. In some embodiments, the co-delivered nucleic acid is configured for protein expression. In some embodiments, the co-delivered nucleic acid is configured for knock-out or knock-down of a gene. In some embodiments, the co-delivered nucleic acid comprises, including is, a DNA, RNA (such as mRNA), siRNA, miRNA, or oligonucleotide (such as an antisense oligonucleotide).

In some embodiments, the co-delivered cargo, such as nucleic acid, is configured to modulate, such as increase, the expression of any one of more of IL-2 (such as membrane bound IL-2), IL-12 (such as membrane bound IL-12), IL-15, IL-21, BCL-2, CXCR4, CCR7, TCF7, FOXO3, FOXp3, or a puromycin resistance gene (such as useful for selective screening of cells receiving delivery of cargo).

In certain aspects, provided herein is a cell population produced according to a method described herein for co-delivery of a plurality of different gene editing complexes and another cargo, such as a polypeptide or a nucleic acid, wherein the cell population comprises about 60% to about 100%, such as at least about any of 60%, 65%, 70%, 75%, 80%, 85%, 90%, or 95%, having both the plurality of different gene editing complexes and the other cargo. In some embodiments, the cell population has not been subjected to further purification and/or selective growth processes following the co-delivery. In some embodiments, the method comprises passing a cell suspension through a material comprising pores therethrough, wherein the cell suspension comprises the plurality of different gene editing complexes and the other cargo. In some embodiments, the cell population comprises about 100,000 cells to about 1×1020 cells. In some embodiments, the produced cell population is about 10% to about 100% of the initial cell population, such as at least about any of 10%, 20%, 30%, 40%, 50%, 60%, 70%, 80%, 90%, or 100%.

In some embodiments, the methods described herein further enable the sequential delivery of cargo, including, e.g., a polypeptide, a nucleic acid molecule, or a gene editing complex (such as targeting another gene or genetic motif), or elements thereof. In some embodiments, the method comprises a first step comprising processing a cell such that a first cargo enters the cell followed by a second step comprising processing the cell such a second cargo enters the cell. In some embodiments, the first and second steps are separated by a rest period. In some embodiments, the first step comprising processing the cell such that the first cargo enters the cell comprises a step of delivering a plurality of different editing complexes. In some embodiments, the rest period comprises between about 0 to about 24 hours. In some embodiments, the rest period comprises between about 0 hours to about 4 hours, between about 4 hours to about 8 hours, between about 8 hours to about 12, between about 12 to about 16, between about 16 to about 20 or between about 20 to about 24 hours. In some embodiments, the rest period comprises between about 1 to about 14 days. In some embodiments, the rest period comprises about 1 day, about 2 days, about 3 days, about 4 days, about 5 days, about 6 days, about 7 days, about 8 days, about 9 days, about 10 days, about 11 days, about 12 days, about 13 days, or about 14 days. In some embodiments, one or more cargos are delivered at each sequential delivery step. In some embodiments, the first step comprising processing a cell such that a first cargo enters the cell is performed prior to cell expansion. In some embodiments, the first step comprising processing a cell such that a first cargo enters the cell is performed prior to cell differentiation or activation.

In some embodiments, the co-delivered cargo (or cargos) comprise, such as is, a polypeptide (such as a protein or a peptide), nucleic acid, compound (such as a small molecule compound), nanoparticle, or virus or virus-like particle. Exemplary nucleic acids include, without limitation, recombinant nucleic acids, DNA, recombinant DNA, cDNA, genomic DNA, RNA, SIRNA, mRNA, saRNA, miRNA, lncRNA, tRNA, shRNA, self-amplifying mRNA, and peptide nucleic acids. In some embodiments, the nucleic acid is homologous to a nucleic acid in the cell. In some embodiments, the nucleic acid is heterologous to a nucleic acid in the cell. In some embodiments, the nucleic acid comprises a transposon, and optionally a sequence encoding a transposase. In some embodiments, the nucleic acid is a plasmid. In some embodiments, the nucleic acid is a therapeutic nucleic acid. In some embodiments, the nucleic acid encodes a therapeutic polypeptide. In some embodiments the nucleic acid encodes a reporter or a selectable marker. Exemplary reporter markers include, without limitation, green fluorescent protein (GFP), red fluorescent protein (RFP), auquorin, beta-galactosidase, Uroporphyrinogen (urogen) III methyltransferase (UMT), and luciferase. Exemplary selectable markers include, without limitation, Blasticidin, G418/Geneticin, Hygromycin B, Puromycin, Zeocin, Adenine Phosphoribosyltransferase, and thymidine kinase. In some embodiments, the nucleic acid modulates expression of a polypeptide in a cell, such as an antisense oligonucleotide or a siRNA.

In some embodiments, the polypeptide comprises a therapeutic protein, antibody, fusion protein, antigen, synthetic protein, reporter marker, or selectable marker. In some embodiments, the polypeptide is a gene-editing protein or nuclease such as a zinc-finger nuclease (ZFN), transcription activator-like effector nuclease (TALEN), mega nuclease, CRE recombinase, FLP recombinase, R recombinase, integrase, or transposase. In some embodiments, the fusion proteins can include, without limitation, chimeric protein drugs such as antibody drug conjugates or recombinant fusion proteins such as proteins tagged with GST or streptavidin. In some embodiments, the co-delivered cargo is a transcription factor. Exemplary transcription factors include, without limitation, Oct5, Sox2, c-Myc, Klf-4, T-bet, GATA3, FoxP3, and RORγt. In some embodiments, the polypeptide comprises a construct, such as a CAR, BITE, or TCR.

In some embodiments, the co-delivered cargo comprises an antigen. An antigen is a substance that stimulates a specific immune response, such as a cell or antibody-mediated immune response. Antigens bind to receptors expressed by immune cells, such as T cell receptors (TCRs), which are specific to a particular antigen. Antigen-receptor binding subsequently triggers intracellular signaling pathways that lead to downstream immune effector pathways, such as cell activation, cytokine production, cell migration, cytotoxic factor secretion, and antibody production. In some embodiments, antigens are derived from foreign sources, such as bacteria, fungi, viruses, or allergens. In some embodiments, antigens are derived from internal sources, such as cancer cells or self-proteins (i.e., self-antigens). Self-antigens are antigens present on an organism's own cells. Self-antigens do not normally stimulate an immune response, but may in the context of autoimmune diseases, such as Type I Diabetes or Rheumatoid Arthritis. In some embodiments, the antigen is a neoantigen. Neoantigens are antigens that are absent from the normal human genome, but are created within oncogenic cells as a result of tumor-specific DNA modifications that result in the formation of novel protein sequences.

In some embodiments, the polypeptide is a reporter or a selectable marker. Exemplary reporter markers include, without limitation, green fluorescent protein (GFP), red fluorescent protein (RFP), auquorin, beta-galactosidase, Uroporphyrinogen (urogen) III methyltransferase (UMT), and luciferase. Exemplary selectable markers include, without limitation, Blasticidin, G418/Geneticin, Hygromycin B, Puromycin, Zeocin, Adenine Phosphoribosyltransferase, and thymidine kinase.

In some embodiments, the polypeptide comprises, such as is, an antibody. In some embodiments, the antibody is a full-length antibody or an antibody fragment. Antibodies for use in the present disclosure include, without limitation, human or humanized antibodies, antibody variants, labeled antibodies, antibody fragments such as Fab or F(ab)₂ fragments, single-domain antibodies, single-chain antibodies, multi-specific antibodies, antibody fusion proteins, and immunoadhesins. The antibodies may be any isotype known in the art, including IgA, IgG, IgE, IgD, or IgM.

In some embodiments, the co-delivered cargo comprises, such as is, a small molecule. Exemplary small molecules include, without limitation, fluorescent markers, dyes, pharmaceutical agents, metabolites, adjuvants, or radionucleotides. In some embodiments, the pharmaceutical agent is a therapeutic drug and/or cytotoxic agent. In some embodiments, the adjuvant includes, without limitation, CpG, oligodeoxynucleotide, R848, lipopolysaccharide (LPS), rhIL-2, anti-CD40 or CD40L, IL-12, cyclic di-nucleotides, and stimulator of interferon genes (STING) agonists.

In some embodiments, the co-delivered cargo comprises a nanoparticle. Examples of nanoparticles include gold nanoparticles, quantum dots, carbon nanotubes, nanoshells, dendrimers, and liposomes. In some embodiments, the nanoparticle contains a therapeutic molecule. In some embodiments, the nanoparticle contains a nucleic acid, such as mRNA. In some embodiments, the nanoparticle contains a label, such as a fluorescent or radioactive label.

In some embodiments, the co-delivered cargo comprises, such as is, a fluorescently tagged molecule, such as a molecule tagged with a fluorochrome such as pacific blue, Alexa 288, Cy5, or cascade blue. In some embodiments, the co-delivered cargo is a radionucleotide, dextran particle, magnetic bead, or impermeable dye. In some embodiments, the co-delivered cargo is a 3 kDa dextran particle labeled with PacBlue. In some embodiments, the co-delivered cargo is a 10 kDa dextran particles labeled with Alexa488. In some embodiments, the co-delivered cargo is a 10 kDa dextran particles labeled with Alexa647. In some embodiments, the co-delivered cargo is a small molecule fluorophore tagged protein. In some embodiments, the co-delivered cargo is a small molecule tagged with Alexa647.

In some embodiments, the co-delivered cargo comprises, such as is, a virus or virus-like particle. In some embodiments, the virus is a retrovirus. In some embodiments, the virus is a therapeutic virus. In some embodiments, the virus is an oncolytic virus. In some embodiments, the virus or virus-like particle contains nucleic acid encoding a therapeutic molecule, such as a therapeutic polypeptide.

### D. Cell Suspensions and Cells

The method provided herein comprise subjecting a cell to a mechanical deformation, such as passing a cell through a pore of a material comprising pores therethrough. To perform such methodology, cells are generally processed in a cell suspension.

In some embodiments, the cell suspension has a volume of about 10 μL to about 5 L, such as any of 100 μL, 500 μL, 1 mL, 5 mL, 10 mL, 50 mL, 100 mL, 200 mL, 300 mL, 400 mL, 500 mL, 600 mL, 700 mL, 800 mL, 900 mL, 1 L, 2 L, 3 L, 4 L, or 5 L. In some embodiments, the cell suspension has a volume of at least about 10 μL, such as at least about any of 100 μL, 500 μL, 1 mL, 5 mL, 10 mL, 50 mL, 100 mL, 200 mL, 300 mL, 400 mL, 500 mL, 600 mL, 700 mL, 800 mL, 900 mL, 1 L, 2 L, 3 L, 4 L, or 5 L. Accordingly, in some embodiments, the methods comprise selecting a mechanical deformation technique, or component thereof, suitable for processing such volume.

It is conceived that, in some embodiments, the cargo to be delivered is a limited resource, such as based on availability and/or cost, and thus the volume of the cell suspension can be modulated accordingly to ensure a certain concentration of the cargo is present in the cell suspension for delivery. In some embodiments, the concentration of the cargo present in the cell suspension for delivery is between about 0.01 mg/mL to about 10 mg/mL, such as any of about 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. In some embodiments, the concentration of the cargo present in the cell suspension for delivery is between about 0.05 mg/mL to about 2 mg/mL. In some embodiments, the concentration of the cargo present in the cell suspension for delivery is at least about 0.01 mg/mL, such as at least about 0.01 mg/mL, 0.02 mg/mL, 0.05 mg/mL, 0.1 mg/mL, 0.2 mg/mL, 0.5 mg/mL, 1.0 mg/mL, 2.0 mg/mL, 3 mg/mL, 4 mg/mL, 5 mg/mL, or 10 mg/mL. In some embodiments, the concentration of the cargo present in the cell suspension for delivery is between about 0.01 mg/mL to about 10 mg/mL, such as any of about 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and the cell density is about 20 million cells/mL to about 500 million cells/mL.

In some embodiments, the cell suspension has a cell density of at least about 100 cells/mL, such as at least about 200 cells/mL, 300 cells/mL, 400 cells/mL, 500 cells/mL, 600 cells/mL, 700 cells/mL, 800 cells/mL, 900 cells/mL, 1,000 cells/mL, 1,250 cells/mL, 1,500 cells/mL, 1,750 cells/mL, 2,000 cells/mL, 2,250 cells/mL, 2,500 cells/mL, 2,750 cells/mL, 3,000 cells/mL, 3,250 cells/mL, 3,500 cells/mL, 3,750 cells/mL, 4,000 cells/mL, 4,250 cells/mL, 4,500 cells/mL, 4,750 cells/mL, 5,000 cells/mL, 10,000 cells/mL, 50,000 cell/mL, 100,000 cell/mL, 1,000,000 cell/mL, 5,000,000 cells/mL, 10,000,000 cells/mL, 25,000,000 cells/mL, 50,000,000 cells/mL, 100,000,000 cells/mL, 150,000,000 cells/mL, or 200,000,000 cells/mL. In some embodiments, the cell suspension has a cell density of about 1,000,000 cells/mL to about 200,000,000 cells/mL. In some embodiments, the cell suspension has a cell density of about 20,000,000 cells/mL to about 100,000,000 cells/mL.

In some embodiments, the cell is a cell obtained from, or derived from, an individual. In some embodiments, the individual is a mouse. In some embodiments, the individual is a non-human primate. In some embodiments, the individual is a human. In some embodiments, the individual is a mouse, dog, cat, horse, rat, goat or rabbit.

In some embodiments, the cell suspension substantially contains a single cell type (e.g., at least about 90% of the cells in a cell suspension are of a single cell type, e.g., T cells). In some embodiments, the cell suspension comprises a plurality of cell types, e.g., as found in cells from whole blood, lymph, and/or peripheral blood mononuclear cells (PBMCs). In some embodiments, the cell suspension comprises a purified cell population.

In some embodiments, the cell (or cells) is a somatic cell, or a stem cell, or a derivative thereof. In some embodiments, the cell is an immune cell. In some embodiments, the cell (or cells) is a PBMC, or a derivative thereof. In some embodiments, the cell (or cells) is a T cell, NK cell, monocyte, B cell, or dendritic cell (or mixture thereof). In some embodiments, the cell is an unstimulated T cell. In some embodiments, the cell is a stimulated T cell. In some embodiments, the cell is a stem cell. In some embodiments, the cell is a human stem cell. In some embodiments, the cell is a hematopoietic stem cell (HSC). In some embodiments, the cell is a mesenchymal stem cell (MSC). In some embodiments, the cell is an induced pluripotent stem cell. In some embodiments, the cell is a cancer cell line, such as a HEK293.

In some embodiments, the cell suspension comprises a buffer. Such buffer may, in certain aspects, serve as a vehicle to enable processing of cells as described herein. As the buffer will contain cells, buffers envisioned herein are compatible with the cell being processed, such as, e.g., to avoid lysis and/or undesirable alterations of said cell. For example, in some embodiments, the buffer has a desired osmolarity, salt concentration, serum content, cell concentration, and pH. In some embodiments, the buffer is a physiological saline solution or a physiological medium other than blood.

In some embodiments, the cell suspension comprises an aqueous solution. In some embodiments, the aqueous solution comprises cell culture medium, PBS, salts, sugars, growth factors, animal derived products, bulking materials, surfactants, lubricants, vitamins, proteins, chelators, and/or an agent that impacts actin polymerization. In some embodiments, the cell culture medium is DMEM, OptiMEM, EVIDM, RPMI, or X-VIVO. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

In some embodiments, the cells can be incubated in one or more solutions that aid in the delivery of the cargo to the interior of the cell. In some embodiments, the aqueous solution comprises an agent that impacts actin polymerization. In some embodiments, the agent that impacts actin polymerization is Latrunculin A, Cytochalasin, and/or Colchicine.

### E. Efficacy of Delivery, Co-Delivery, and Cell Viability

The methods of delivery described herein, provide, in certain aspects, a degree of delivery efficacy and/or cell viability. For example, the methods described herein provide a desired yield of engineered cells that are viable for downstream uses.

In some embodiments, following the subjecting a cell suspension to a mechanical deformation, such as passing cells through a material having pores therethrough, at least about 25%, such as at least about any of 30%, 35%, 40%, 45%, 50%, 55%, 60%, 65%, 70%, 75%, 80%, 85%, 90%, or 95%, of the cells receive at least one, including two or more or all, of a plurality of different gene editing complexes.

In some embodiments, following the subjecting a cell suspension to a mechanical deformation, such as passing cells through a material having pores therethrough, at least about 25%, such as at least about any of 30%, 35%, 40%, 45%, 50%, 55%, 60%, 65%, 70%, 75%, 80%, 85%, 90%, or 95%, of the cells are edited according to the design of at least one of the plurality of different gene editing complexes.

In some embodiments, following the subjecting a cell suspension to a mechanical deformation, such as passing cells through a material having pores therethrough, at least about 25%, such as at least about any of 30%, 35%, 40%, 45%, 50%, 55%, 60%, 65%, 70%, 75%, 80%, 85%, 90%, or 95%, of cells are viable.

In some embodiments, following the subjecting a cell suspension to a mechanical deformation, such as passing cells through a material having pores therethrough, at least about 25%, such as at least about any of 30%, 35%, 40%, 45%, 50%, 55%, 60%, 65%, 70%, 75%, 80%, 85%, 90%, or 95%, of the cells that comprise at least one edit according to the design of at least one of the plurality of different gene editing complexes also contain a co-delivered cargo, such as a nucleic acid.

### F. Additional Aspects of the Methods Provided Herein

The present disclosure further encompasses both upstream and downstream processes useful or associated with the delivery methods described herein. For example, in some embodiments, the method further comprises obtaining a cell or cells, such as from an individual. In some embodiments, the method further comprises obtaining a cell suspension comprising a cell or cells. In some embodiments, the method further comprises preparing a mechanical deformation technique, or component thereof, such as a material having pores therethrough, for a method of delivery as described herein (such as washing said material). In some embodiments, the method further incubating a cell or cells processed with a mechanical deformation such that the cell or cells close the pores made therein. In some embodiments, the method further comprises storing a cell suspension or cells subjected to the delivery methods described herein. In some embodiments, the method further comprises validating delivery of cargo to a cell or cells following performing a delivery method described herein.

### G. Example Methods of Delivery

Provided herein, in certain aspects, is a method of delivering to a cell, such as an unstimulated T cell, a plurality of different gene editing complexes targeting a gene or genetic motif, the method comprising: passing a cell suspension through a material having pores therethrough, the cell suspension comprising the cell, wherein the pores have an average pore size (as measured by the largest crossing dimension, e.g., diameter) of about 3 μm to about 6 μm, such as about any of 3 μm, 3.5 μm, 4 μm, 4.5 μm, 4 μm, 4.5 μm, 5 μm, 5.5 μm, or 6 μm, and wherein the passing the cell suspension through the material having pores therethrough is performed at a pressure of about 1 psi to about 20 psi, such as about any of 7 psi, 8 psi, 9 psi, 10 psi, 11 psi, 12 psi, 13 psi, 14 psi, or 15 psi. In some embodiments, the plurality of different gene editing complexes, or elements thereof, are provided in proximity to the cell prior to, during, or directly after (such as within about one minute) passing the cell suspension through the material having pores therethrough. In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid. In some embodiments, the material having pores therethrough is a microsieve (e.g., a silicon microsieve). In some embodiments, the material having pores therethrough is a polymer-based track-etched filter (e.g., a polycarbonate track-etched filter).

Provided herein, in certain aspects, is a method of delivering to a cell a plurality of different gene editing complexes targeting a gene or genetic motif, the method comprising: passing a cell suspension through a polymer-based material having pores therethrough, such as a track-etched polymer-based filter (e.g., a polycarbonate track-etched filter), the cell suspension comprising the cell. In some embodiments, the pores have an average pore size (as measured by the largest crossing dimension, e.g., diameter) of about 3 μm to about 15 μm, such as about any of 3 μm, 3.5 μm, 4 μm, 4.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, or 15 μm. In some embodiments, the passing the cell suspension through the material having pores therethrough is performed at a pressure of about 1 psi to about 20 psi, such as about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, or 20 psi. In some embodiments, the plurality of different gene editing complexes, or elements thereof, are provided in proximity to the cell prior to, during, or directly after (such as within about one minute) passing the cell suspension through the polymer-based material having pores therethrough, such as a track-etched polymer-based filter (e.g., a polycarbonate track-etched filter). In some embodiments, the cell suspension comprises the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid.

Provided herein, in certain aspects, is a method of delivering to a cell, such as an activated T cell, a plurality of different gene editing complexes targeting a gene or genetic motif, the method comprising: passing a cell suspension through a material having pores therethrough, the cell suspension comprising the cell, wherein the pores have an average pore size (as measured by the largest crossing dimension, e.g., diameter) of about 6 μm to about 9 μm, such as about any of 6 μm, 6.5 μm, 7 μm, 7.5 μm, 8 μm, 8.5 μm, or 9 μm, and wherein the passing the cell suspension through the material having pores therethrough is performed at a pressure of about 1 psi to about 20 psi, such as about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, or 20 psi. In some embodiments, the plurality of different gene editing complexes, or elements thereof, are provided in proximity to the cell prior to, during, or directly after (such as within about one minute) passing the cell suspension through the material having pores therethrough. In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid. In some embodiments, the material having pores therethrough is a microsieve (e.g., a silicon microsieve). In some embodiments, the material having pores therethrough is a track-etched filter (e.g., a polycarbonate track-etched filter).

Provided herein, in certain aspects, is a method of delivering to a cell, such as human stem cell (HSC), a plurality of different gene editing complexes targeting a gene or genetic motif, the method comprising: passing a cell suspension through a material having pores therethrough, the cell suspension comprising the cell, wherein the pores have an average pore size (as measured by the largest crossing dimension, e.g., diameter) of about 3 μm to about 6 μm, such as about any of 3 μm, 3.5 μm, 4 μm, 4.5 μm, 4 μm, 4.5 μm, 5 μm, 5.5 μm, or 6 μm, and wherein the passing the cell suspension through the material having pores therethrough is performed at a pressure of about 1 psi to about 20 psi, such as about any of 7 psi, 8 psi, 9 psi, 10 psi, 11 psi, 12 psi, 13 psi, 14 psi, or 15 psi. In some embodiments, the plurality of different gene editing complexes, or elements thereof, are provided in proximity to the cell prior to, during, or directly after (such as within about one minute) passing the cell suspension through the material having pores therethrough. In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid. In some embodiments, the material having pores therethrough is a microsieve (e.g., a silicon microsieve). In some embodiments, the material having pores therethrough is a track-etched filter (e.g., a polycarbonate track-etched filter).

Provided herein, in certain aspects, is a method of delivering to cells, such as PBMCs, a plurality of different gene editing complexes targeting a gene or genetic motif, the method comprising: passing a cell suspension through a material having pores therethrough, the cell suspension comprising the cells, wherein the pores have an average pore size (as measured by the largest crossing dimension, e.g., diameter) of about 3 μm to about 6 μm, such as about any of 3 μm, 3.5 μm, 4 μm, 4.5 μm, 4 μm, 4.5 μm, 5 μm, 5.5 μm, or 6 μm, and wherein the passing the cell suspension through the material having pores therethrough is performed at a pressure of about 1 psi to about 20 psi, such as about any of 7 psi, 8 psi, 9 psi, 10 psi, 11 psi, 12 psi, 13 psi, 14 psi, or 15 psi. In some embodiments, the plurality of different gene editing complexes, or elements thereof, are provided in proximity to the cell prior to, during, or directly after (such as within about one minute) passing the cell suspension through the material having pores therethrough. In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid. In some embodiments, the material having pores therethrough is a microsieve (e.g., a silicon microsieve). In some embodiments, the material having pores therethrough is a track-etched filter (e.g., a polycarbonate track-etched filter).

Provided herein, in certain aspects, is a method of delivering to a cell, such as an iPSC, a plurality of different gene editing complexes targeting a gene or genetic motif, the method comprising: passing a cell suspension through a material having pores therethrough, the cell suspension comprising the cell, wherein the pores have an average pore size (as measured by the largest crossing dimension, e.g., diameter) of about 7 μm to about 12 μm, such as about any of 7 μm, 7.5 μm, 8 μm, 8.5 μm, 9 μm, 9.5 μm, 10 μm, 10.5 μm, 11 μm, 11.5 μm, or 12 μm, and wherein the passing the cell suspension through the material having pores therethrough is performed at a pressure of about 1 psi to about 20 psi, such as about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, or 20 psi. In some embodiments, the plurality of different gene editing complexes, or elements thereof, are provided in proximity to the cell prior to, during, or directly after (such as within about one minute) passing the cell suspension through the material having pores therethrough. In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid. In some embodiments, the material having pores therethrough is a microsieve (e.g., a silicon microsieve). In some embodiments, the material having pores therethrough is a track-etched filter (e.g., a polycarbonate track-etched filter).

Provided herein, in certain aspects, is a method of delivering to a cell, such as a HEK293 cell, a plurality of different gene editing complexes targeting a gene or genetic motif, the method comprising: passing a cell suspension through a material having pores therethrough, the cell suspension comprising the cell, wherein the pores have an average pore size (as measured by the largest crossing dimension, e.g., diameter) of about 8 μm to about 12 μm, such as about any of 8 μm, 8.5 μm, 9 μm, 9.5 μm, 10 μm, 10.5 μm, 11 μm, 11.5 μm, or 12 μm, and wherein the passing the cell suspension through the material having pores therethrough is performed at a pressure of about 1 psi to about 20 psi, such as about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, or 20 psi. In some embodiments, the plurality of different gene editing complexes, or elements thereof, are provided in proximity to the cell prior to, during, or directly after (such as within about one minute) passing the cell suspension through the material having pores therethrough. In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid. In some embodiments, the material having pores therethrough is a microsieve (e.g., a silicon microsieve). In some embodiments, the material having pores therethrough is a track-etched filter.

Provided herein, in certain aspects, is a method of delivering to a cell a plurality of different gene editing complexes targeting a gene or genetic motif, the method comprising: passing a cell suspension through a microsieve (e.g., a silicon microsieve) comprising a material having pores therethrough, wherein the cell suspension comprising the cell. In some embodiments, the pores have an average pore size (as measured by the largest crossing dimension, e.g., diameter) of about 3 μm to about 15 μm, such as about any of 3 μm, 3.5 μm, 4 μm, 4.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, or 15 μm. In some embodiments, the passing the cell suspension through the microsieve (e.g., a silicon microsieve) is performed at a pressure of about 1 psi to about 20 psi, such as about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, or 20 psi. In some embodiments, the plurality of different gene editing complexes, or elements thereof, are provided in proximity to the cell prior to, during, or directly after (such as within about one minute) passing the cell suspension through the microsieve (e.g., a silicon microsieve). In some embodiments, the cell suspension comprising the plurality of different gene editing complexes, or elements thereof. In some embodiments, the method further comprises co-delivering another cargo, such as a polypeptide, nucleic acid, or a complex comprising a polypeptide and a nucleic acid. In some embodiments, the cell suspension comprises another cargo, such as a polypeptide, nucleic acid, or a complex comprising a polypeptide and a nucleic acid.

Provided herein, in certain aspects, is a method of co-delivering to a cell a plurality of different gene editing complexes targeting a gene or genetic motif and another cargo, such as a nucleic acid, the method comprising: passing a cell suspension through a material having pores therethrough, wherein the cell suspension comprising the cell. In some embodiments, the pores have an average pore size (as measured by the largest crossing dimension, e.g., diameter) of about 3 μm to about 15 μm, such as about any of 3 μm, 3.5 μm, 4 μm, 4.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, or 15 μm. In some embodiments, the passing the cell suspension through the material having pores therethrough is performed at a pressure of about 1 psi to about 20 psi, such as about any of 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, or 20 psi. In some embodiments, the plurality of different gene editing complexes, or elements thereof, an/d or the other cargo are provided in proximity to the cell prior to, during, or directly after (such as within about one minute) passing the cell suspension through the material having pores therethrough. In some embodiments, the cell suspension comprising the plurality of different gene editing complexes, or elements thereof, and/or the other cargo. In some embodiments, the material having pores therethrough is a microsieve (e.g., a silicon microsieve). In some embodiments, the material having pores therethrough is a track-etched filter (e.g., a polycarbonate track-etched filter). In some embodiments, following the passing the cell suspension through the material having pores therethrough, at least about 25%, such as at least about any of 30%, 35%, 40%, 45%, 50%, 55%, 60%, 65%, 70%, 75%, 80%, 85%, 90%, or 95%, of the cells that comprise at least one edit according to the design of at least one of the plurality of different gene editing complexes also contain the co-delivered cargo, such as a nucleic acid.

### III. Systems, Kits, and Components Useful for the Methods Taught Herein

In other aspects, the present disclosure provides systems, kits, components, and compositions (such as consumables) useful for the methods described herein. For example, provided herein is a system comprising a device for subjecting the cell suspension to a mechanical deformation, such as a material having pores therethrough, wherein the subjecting the cell suspension to the process for mechanical deformation allows the plurality of different gene editing complexes to enter the cell, and a cell suspension comprising a cell. In some embodiments, the cell suspension, or another composition of a system or kit provided herein, further comprises a plurality of gene editing complexes targeting a gene or genetic motif. In some embodiments, the cell suspension, or another composition of a system or kit provided herein, further comprises another cargo, such as another nucleic acid, for co-delivery to a cell. In some embodiments, the components of the systems or kits may come in separate containers. In some embodiments, the system or kit comprises instructions for use according the methods described herein.

### IV. Sequences

Provided herein are sequences useful for the present disclosure.

TABLE 1
CRISPR gRNA sequences

| SEQ ID | SEQ | Base Sequence |
|---|---|---|
| NO.: | information | 5′→3′ |
| 1 | B2M_s1 | CUGAAUCUUUGGAGUACCUG |
| 2 | B2M_s2 | UCACGUCAUCCAGCAGAGAA |
| 3 | B2M_v1 | CUUGUCAAGGCUAUUGGUCA |
| 4 | B2M_v2 | GCUACUCUCUCUUUCUGGCC |
| 5 | TRAC_s1 | GUCAGGGUUCUGGAUAUCUG |
| 6 | TRAC_s2 | GCUGGUACACGGCAGGGUCA |
| 7 | TRAC_i 1 | AGAGUCUCUCAGCUGGUACA |
| 8 | TRAC_i 2 | UGUGCUAGACAUGAGGUCUA |

SEQ ID NO. 9: GFP expression construct
AUGGUGAGCAAGGGCGAGGAGCUGUUCACCGGGGUGGUGCCCAUCCUGG
UCGAGCUGGACGGCGACGUAAACGGCCACAAGUUCAGCGUGUCCGGCGA
GGGCGAGGGCGAUGCCACCUACGGCAAGCUGACCCUGAAGUUCAUCUGC
ACCACCGGCAAGCUGCCCGUGCCCUGGCCCACCCUCGUGACCACCCUGA
CCUACGGCGUGCAGUGCUUCAGCCGCUACCCCGACCACAUGAAGCAGCA
CGACUUCUUCAAGUCCGCCAUGCCCGAAGGCUACGUCCAGGAGCGCACC
AUCUUCUUCAAGGACGACGGCAACUACAAGACCCGCGCCGAGGUGAAGU
UCGAGGGCGACACCCUGGUGAACCGCAUCGAGCUGAAGGGCAUCGACUU
CAAGGAGGACGGCAACAUCCUGGGGCACAAGCUGGAGUACAACUACAAC
AGCCACAACGUCUAUAUCAUGGCCGACAAGCAGAAGAACGGCAUCAAGG
UGAACUUCAAGAUCCGCCACAACAUCGAGGACGGCAGCGUGCAGCUCGC
CGACCACUACCAGCAGAACACCCCCAUCGGCGACGGCCCCGUGCUGCUG
CCCGACAACCACUACCUGAGCACCCAGUCCGCCCUGAGCAAAGACCCCA
ACGAGAAGCGCGAUCACAUGGUCCUGCUGGAGUUCGUGACCGCCGCCGG
GAUCACUCUCGGCAUGGACGAGCUGUACAAGUAA

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1: Mechanical Deformation Assisted Complex Delivery and Genome Editing in T Cells Using Low-Porosity Microsieves

This example demonstrates safe and efficient delivery of CRISPR/Cas9 RNPs in human T cells subjected to mechanical deformation forces via the use of low-porosity microsieves.

Cryopreserved human PBMCs were thawed in full growth RPMI media (RPMI 1640 media with 10% HI FBS and 1% Pen/Strep), and T cells were isolated using a bead-based negative isolation kit (STEMCELL, Vancouver, CA). Cells were rested at 2×10⁶ cells/mL for 30 minutes in complete T-cell media containing X-VIVO™ 15 (Lonza, Basel, Switzerland), 55 μM BME (Gibco, Waltham, MA), human AB serum (5%, Sigma Aldrich), L-glutamine (2 mM), MEM non-essential amino acids (1×), and supported with recombinant human IL-2 (100 U/mL), IL-7 (10 ng/ml), and IL-15 (10 ng/mL). Cas9 RNPs specific to the beta-2 microglobulin (B2M) gene were pre-complexed using Cas9 protein and in-house designed guides (Integrated DNA Technologies, Coralville, IA) (SEQ ID NOs: 1-4, Table 1) at a 2.5:1 molar ratio of guide:Cas9 and allowed to complex at room temperature for 10 minutes, then stored on ice prior to use.

T cells were mixed with 100 ug/ml RNP and 100 ug/ml 3 kDa Cascade Blue dextran (Thermo, Waltham, MA) at a final concentration of 20M/ml in Optimem (Gibco). T cells were then subjected to mechanical deformation by passing the cells through low-porosity microsieves (Aquamarijn) containing pore diameters of 4 μm or 5 μm at a pressure of 5, 7, or 10 psi. Pressure was exerted on T cells during passage through the low-porosity microsieves via a pressure regulator containing a buffering reservoir to maintain stable pressure throughout use. As a control, T cells with no contact with RNP complexes (“No contact”), and T cells in contact with RNP complexes (“endo”), were compared to T cells that were mechanically deformed. Additionally, a control group of T cells which contained no RNP complexes was subjected to mechanical deformation (“mechanoporated” (MP)) through a 4 μm or 5 μm diameter low-porosity microsieve at 7 psi. T cells were cultured and washed at 37° C. for 48 hours in complete T cell media following mechanical deformation before conducting cell viability and genome editing efficiency analyses via flow cytometry.

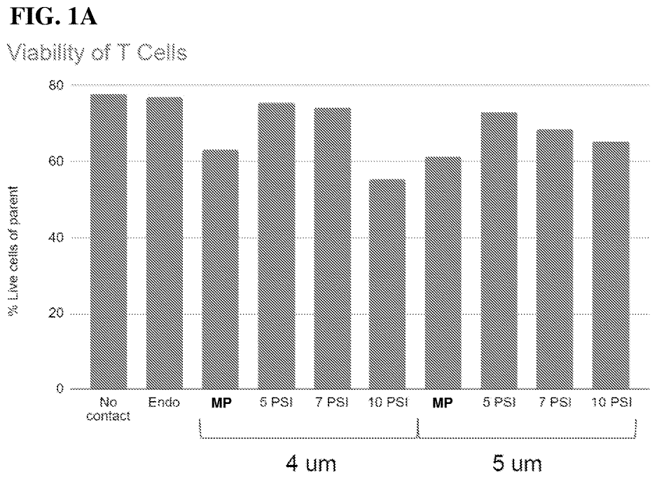

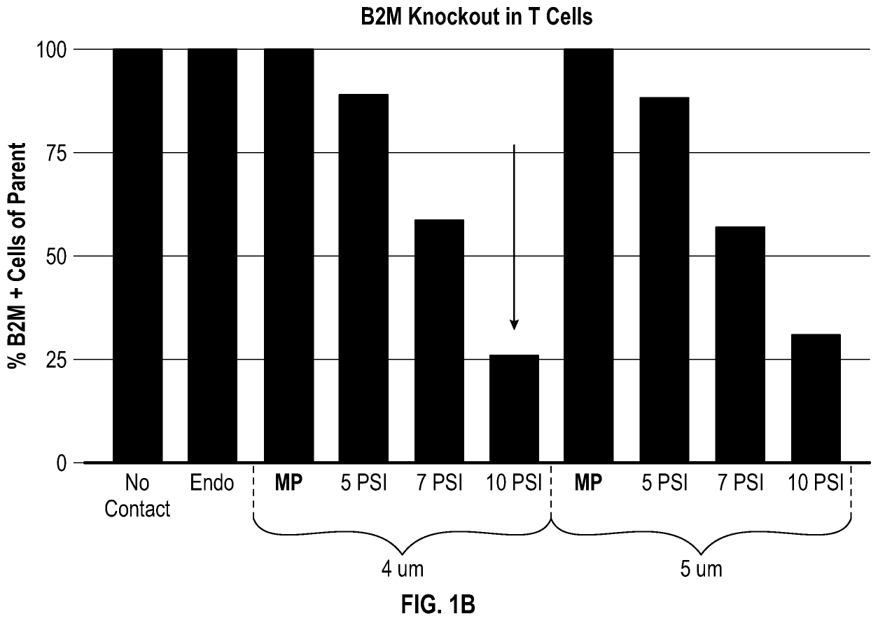

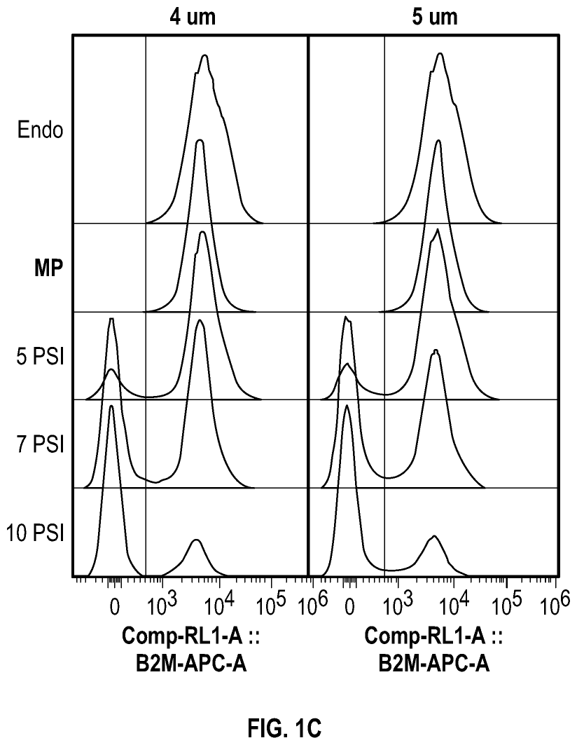

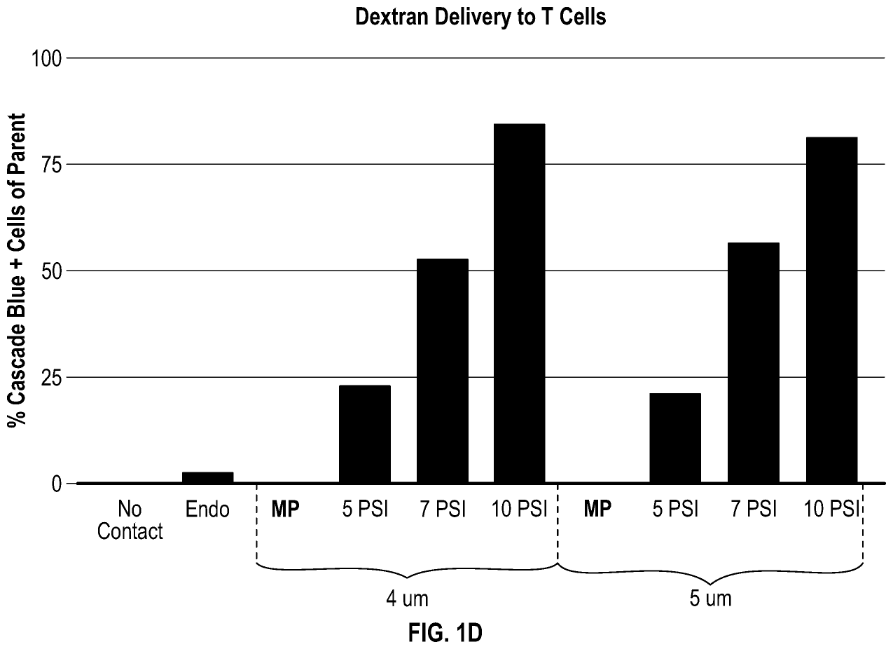

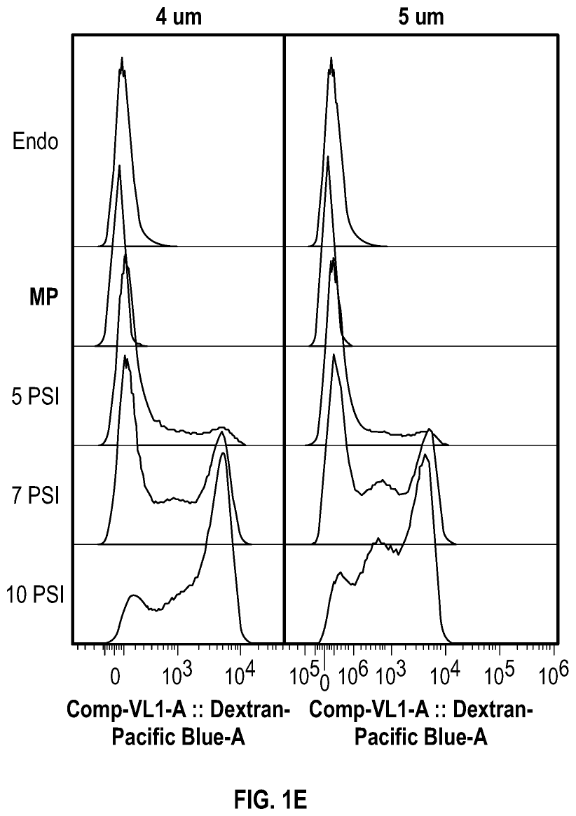

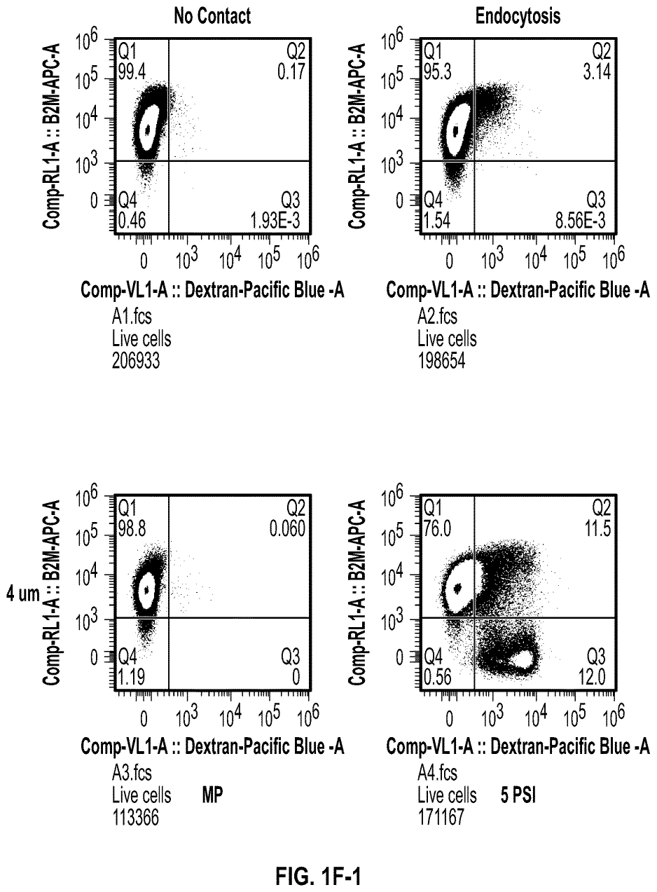

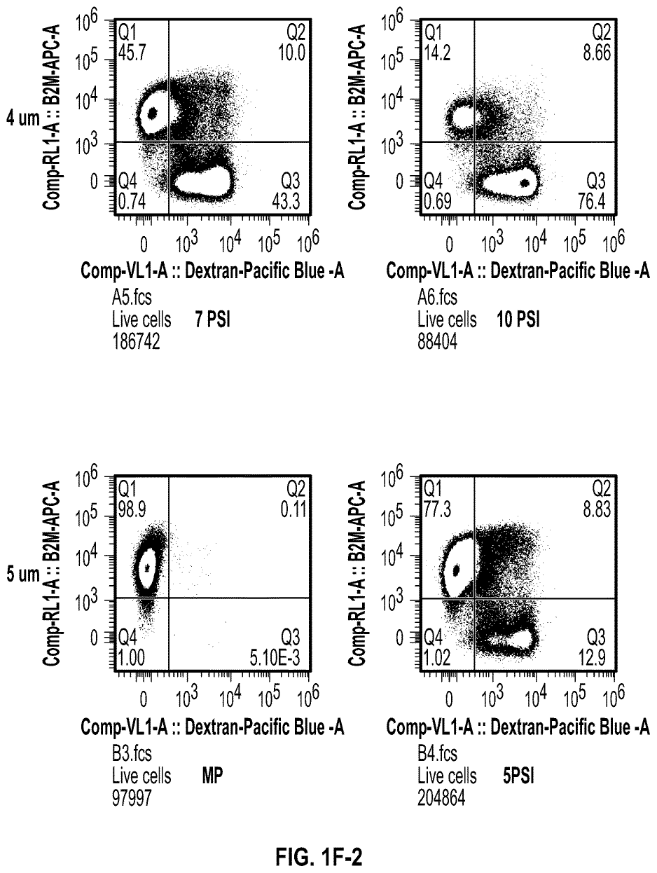

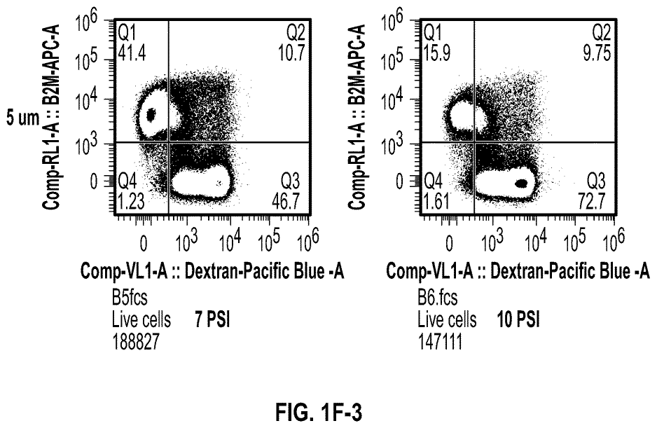

A cell viability analysis was conducted following mechanical deformation assisted genome editing by staining cells with the Live/Dead Near-infrared (Thermo) stain and quantified using flow cytometry. As shown in FIG. 1A, cell viability was only modestly affected following mechanical deformation assisted genome editing. Given that the slight reduction in cell viability was observed in both no contact conditions, this reduction is likely due to the forces exerted on the T cells during mechanical deformation, rather than toxicity caused by RNP derived genome editing.

To assess genome editing at the B2M locus, T cells were immunolabelled with an anti-human β2-microglobulin (B2M) antibody (BioLegend, 395712) and fluorescence was measured by flow cytometry. As shown in FIGS. 1B-1C, genome editing efficiency increased as the pressure in which the T cells were pushed through the low-porosity microsieve increased. The greatest editing efficiency was observed at the 10-psi condition when using both a 4 μm or 5 μm low-porosity microsieve. Genome editing was not observed in “Endo” control cells, suggesting that RNP editing complexes are not delivered into cells without an external stimulus such as mechanical deformation of the plasma membrane.

To monitor RNP delivery and simulate delivery of large polynucleotide cassettes for on-target integration events, fluorescently labeled dextran (3 kDa Cascade Blue dextran) was delivered to T cells simultaneously with RNP complexes. As shown in FIGS. 1D-1E, dextran was effectively delivered to T cells following mechanical deformation. Consistent with the genome editing results shown in FIGS. 1B-1C, the 10-psi condition for both a 4 μm or 5 μm low-porosity microsieve resulted in the greatest percentage of Cascade Blue positive T cells.

A flow cytometry analysis was conducted to compare the delivery of dextran and B2M editing (FIG. 1F). Results were consistent between the 4 μm and 5 μm diameter low-porosity microsieve conditions. As the pressure exerted on the T cells was increased from 5 to 10 psi, a greater percentage of T cells displayed dextran fluorescence and a concomitant decrease in B2M expression was observed. T cells that experienced a force of 5 psi during transit through the 4 μm low-porosity microsieve successfully edited the B2M locus in 12% of cells (Q3), while T cells that experienced a force of 10 psi during transit through the 4 μm low-porosity microsieve edited the B2M locus in 76.4% of cells (Q3). Similar results are observed when using a 5 μm low-porosity microsieve. Surprisingly, the vast majority of cells that displayed dextran fluorescence also failed to express B2M, suggesting that the efficiency of RNP dependent genome editing was greatly improved following mechanical deformation. For T cells that were passed through a 4 μm diameter low-porosity microsieve at a pressure of 10 psi 76.4% of cells both received dextran and successfully edited the B2M locus (Q3), while only 8.66% of cells received dextran and failed to edit the B2M locus (Q2).

### Example 2: Mechanical Deformation Assisted Complex Delivery and Genome Editing in T Cells Using Track-Etched Filters

This example demonstrates safe and efficient delivery of CRISPR/Cas9 RNPs in human T cells subjected to mechanical deformation forces via the use of track-etched filters.

Human T cells were obtained from frozen PBMCs and prepared for mechanical deformation assisted genome editing as disclosed in Example 1.

T cells were subjected to mechanical deformation by passing the cells through track-etched filters (Sterlitech) containing a pore diameter of 5 μm at a pressure of 5, 7, or 10 psi. Pressure was exerted on T cells during passage through the track-etched filters via a pressure regulator containing a buffering reservoir to maintain stable pressure throughout use. As a control, T cells with no contact with RNP complexes (“No contact”), and T cells in contact with RNP complexes (“endo”), were compared to T cells that were mechanically deformed. Additionally, a control group of T cells which were not exposed to RNP complexes were also subjected to mechanical deformation (“mechanoporated” (MP)) through the 5 μm diameter track-etched filter at 7 psi. T cells were cultured and washed at 37° C. for 48 hours in complete T cell media following mechanical deformation before conducting cell viability and genome editing efficiency analyses via flow cytometry.

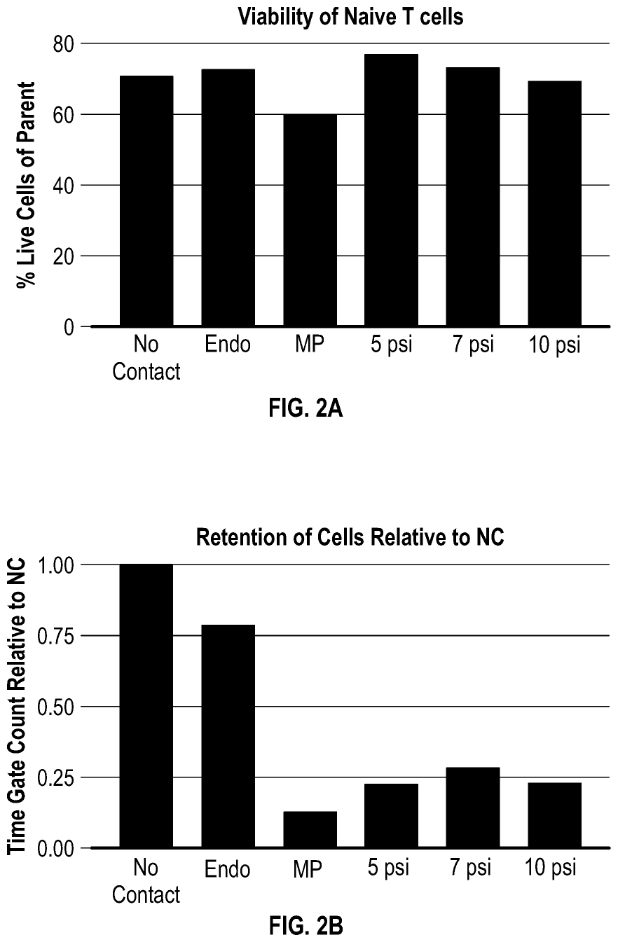

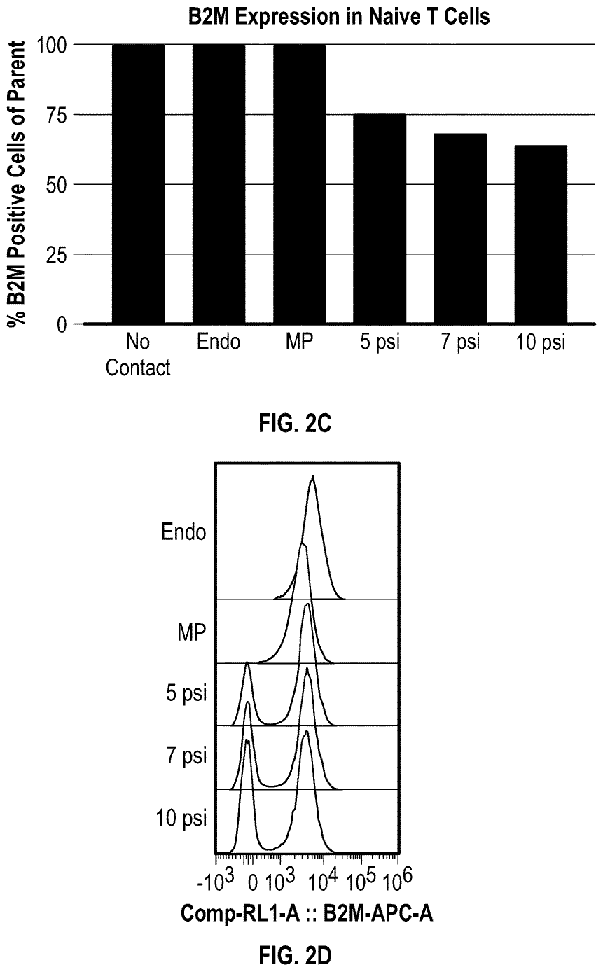

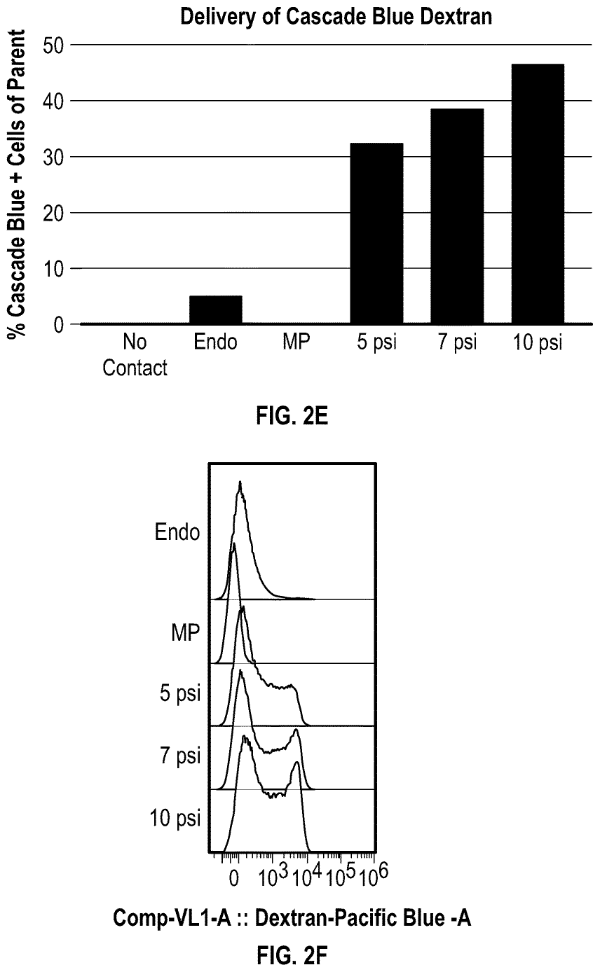

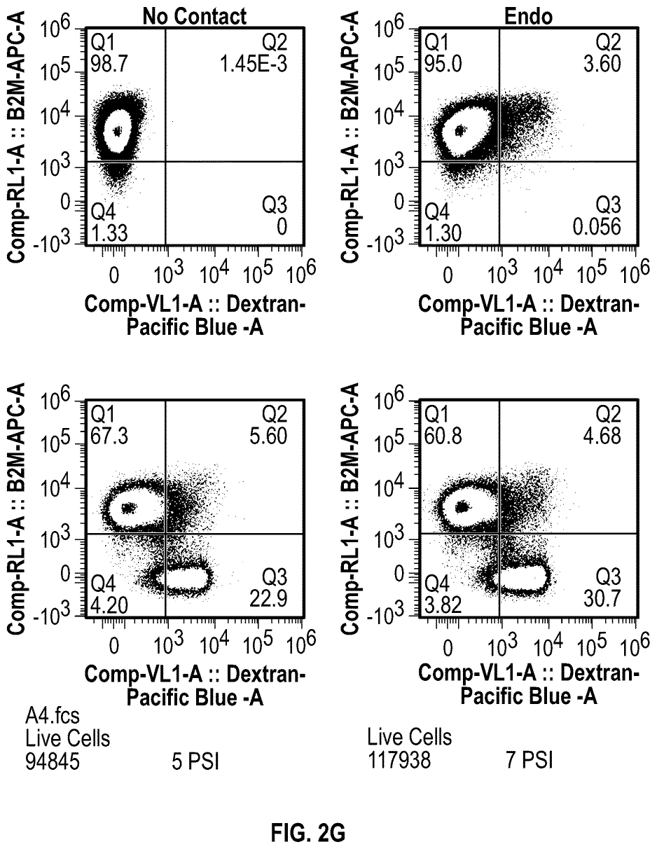

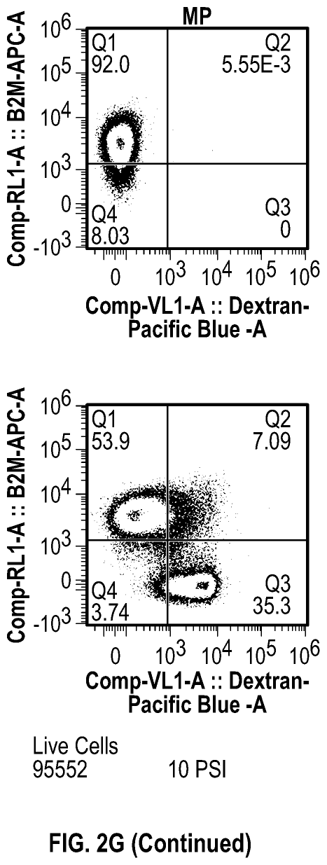

A cell viability analysis was conducted following mechanical deformation assisted genome editing by staining cells with the Live/Dead Near-infrared (Thermo) stain and quantified using flow cytometry. As shown in FIG. 2A, cell viability was only modestly affected following mechanical deformation assisted genome editing. A slight reduction in cell viability was observed in the Empty Boost (EB) control condition, suggesting that cell viability was minimally affected due to the forces exerted on the T cells during mechanical deformation. Passage of T cells through the track-etched filter resulted in a reduced retention of cells compared to the No Contact control cells (FIG. 2B). The relative retention value observed in T cells that were exposed to genome editing complexes and passed through the track-etched filter at pressures of 5 psi, 7 psi, and 10 psi, were similar to the mechanoporated control cells that were not exposed to genome editing complexes.

T cells were immunolabelled with an anti-human β2-microglobulin (B2M) antibody (BioLegend, 395712) to assess genome editing at the B2M locus and fluorescence was measured by flow cytometry. As shown in FIGS. 2C-2D, genome editing efficiency increased as the pressure in which the T cells were pushed through the track-etched filter increased. The greatest editing efficiency was observed at the 10-psi condition when using a 5 μm diameter track-etched filter. As shown in Example 1, genome editing was not observed in “Endo” control cells, suggesting that RNP editing complexes are not delivered into cells without an external stimulus such as mechanical deformation of the plasma membrane.

Fluorescently labeled dextran (3 kDa Cascade Blue dextran) was delivered to T cells simultaneously with RNP complexes to monitor delivery efficiency and simulate delivery of additional cargo such as nucleic acids or proteins. As shown in FIGS. 2E-2F, dextran was effectively delivered to T cells following mechanical deformation. The 10-psi condition using a 5 μm diameter track-etched filter resulted in the greatest percentage of Cascade Blue positive T cells out of the other tested pressures (FIG. 2F).

A flow cytometry analysis was conducted to compare the delivery of dextran and B2M editing (FIG. 2G). As the pressure exerted on the T cells was increased from 5 to 10 psi, a greater percentage of T cells displayed dextran fluorescence and a concomitant decrease in B2M expression was observed. T cells that experienced a pressure of 5 psi, 7 psi, and 10 psi, showed dextran fluorescence and B2M editing in 22.9, 30.7, and 35.3% of cells, respectively (Q3, FIG. 2G). As seen in Example 1, the vast majority of cells that displayed dextran fluorescence also failed to express B2M, suggesting that the efficiency of RNP dependent genome editing was greatly improved following mechanical deformation. For T cells that experienced a pressure of 10 psi during transit through the track-etched filter 35.3% of cells both received dextran and successfully edited the B2M locus (Q3, FIG. 2G), while only 7.09% of cells received dextran and failed to edit the B2M locus (Q2, FIG. 2G).

Together, these results demonstrate that a brief mechanical deformation can safely and effectively deliver RNPs containing a mixture of multiple guide RNAs to T cells. Genome editing at the B2M locus was most efficient in the highest tested pressure condition (10 psi), suggesting that an increased mechanical deformation force both improves delivery of genome editing complexes to T cells and improves the genome editing efficiency of the RNP complexes. Moreover, such techniques provided a high degree of co-delivery, which is a significant finding of the performed methods.

### Example 3: Simultaneous Codelivery of Genome Editing Complexes, Dextran, and mRNA in Various Cell Types

This example further demonstrates the ability of mechanical deformation to successfully codeliver genome editing complexes and nucleic acids to target cells.

Human T cells were obtained from frozen PBMCs and prepared for mechanical deformation assisted genome editing as disclosed in Example 1.

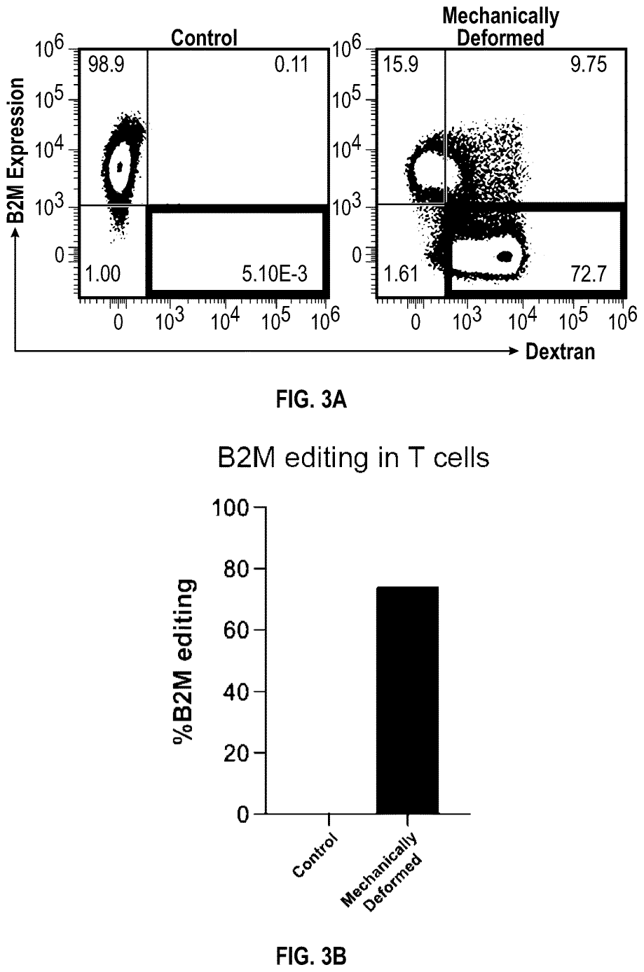

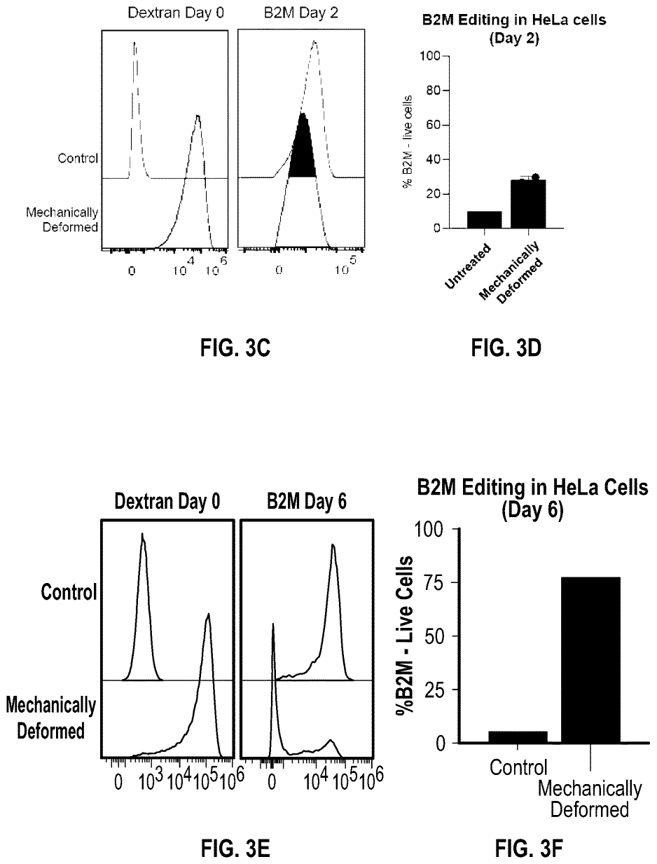

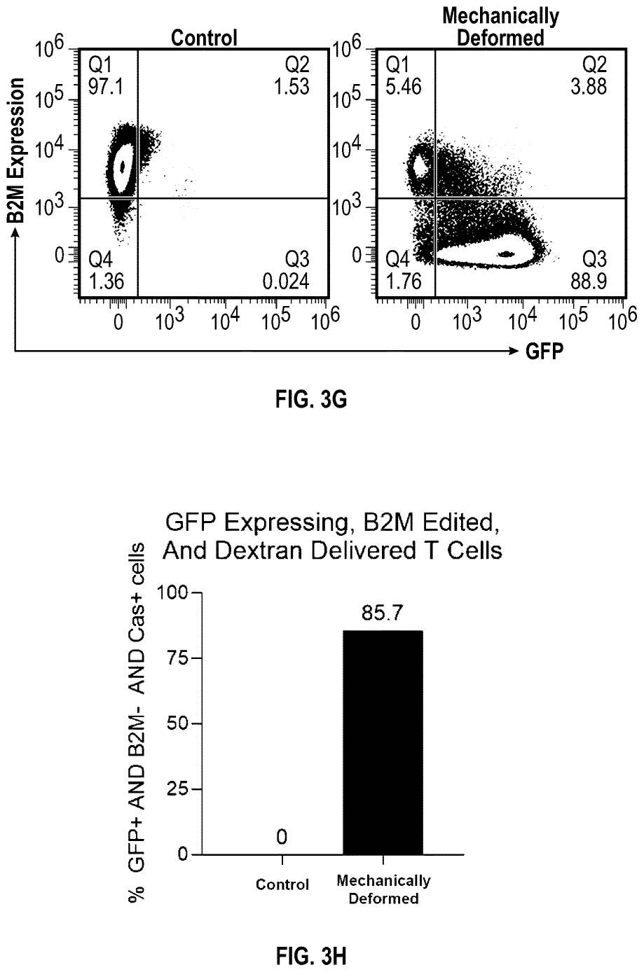

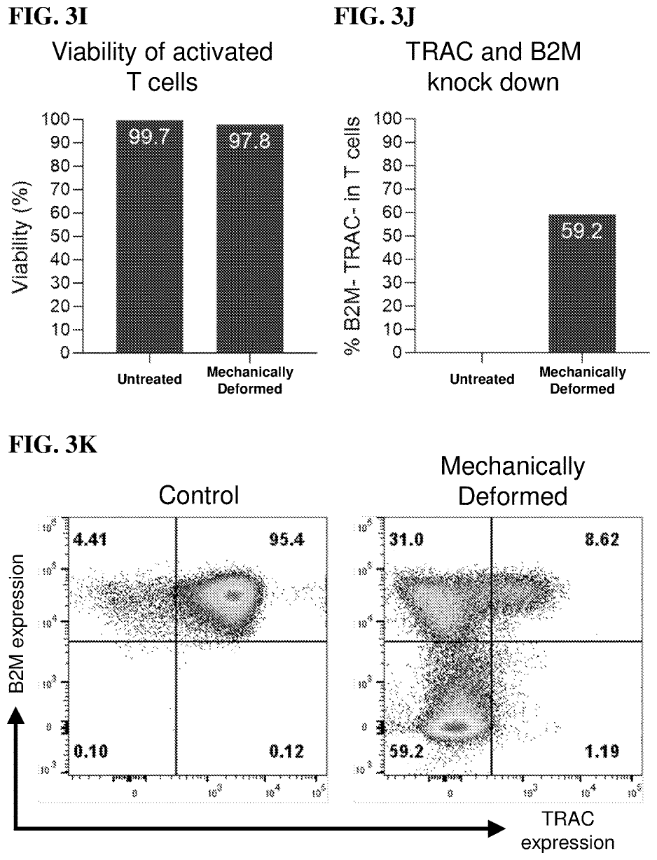

To assess the effectiveness of simultaneously delivering genome editing complexes and simulate delivery of large polynucleotide cassettes for on-target integration events using mechanical deformation, unstimulated T cells were mixed with 100 μg/ml RNP and 100 μg/ml 3 kDa Cascade Blue dextran (Thermo, Waltham, MA) at a final concentration of 20M/ml in Optimem (Gibco). T cells were then subjected to mechanical deformation by passing the cells through low-porosity microsieves (Aquamarijn). T cells were cultured and washed at 37° C. for 48 hours in complete T cell media following mechanical deformation before conducting genome editing efficiency analyses via flow cytometry. To assess genome editing at the B2M locus, T cells were immunolabelled with an anti-human β2-microglobulin (B2M) antibody (BioLegend, 395712) and fluorescence was measured by flow cytometry. As shown in FIG. 3A, control cells which were not subjected to mechanical deformation failed to edit the B2M locus and display Cascade Blue dextran fluorescence (5.10×10⁻³% of cells) while 72.7% of cells which were subjected to mechanical deformation at a pressure of 10 PSI showed low B2M expression and high levels of Cascade Blue dextran fluorescence. As shown in FIG. 3B, 72.7% of cells exposed to mechanical deformation successfully edited the B2M loci while about 0% of control cells successfully edited the B2M loci.

Mechanical deformation assisted delivery of cargo comprising genome editing complexes and Cascade Blue dextran was next tested on Hela cells. Hela cells were mixed with 100 μg/ml RNP targeting the B2M gene (SEQ ID NOs. 1-4) and 100 μg/ml 3 kDa Cascade Blue dextran (Thermo, Waltham, MA) at a final concentration of 20 M/ml in Optimem (Gibco). Hela cells were then subjected to mechanical deformation by passing the cells through low-porosity microsieves (Aquamarijn). Successful delivery of Cascade Blue dextran was quantified by flow cytometry on day 0. Expression of the B2M gene was assessed at days 2 and 6 by immunolabelling Hela cells with an anti-human β2-microglobulin (B2M) antibody (BioLegend, 395712) and measuring fluorescence by flow cytometry. Following exposure to mechanical deformation forces on day 0, HeLa cells successfully displayed high levels of Cascade Blue dextran fluorescence while control cells displayed low levels of Cascade Blue dextran fluorescence (FIG. 3C and FIG. 3E). At day 2 following exposure of the Hela cells to the RNPs targeting the B2M gene and mechanical deformation forces, only about 28% of HeLa cells displayed low levels of B2M expression (FIGS. 3C, 3D). Interestingly, at day 6 following exposure about 78% of HeLa cells displayed low levels of B2M expression (FIGS. 3E, 3F). The surprising delay in knockdown of B2M expression in HeLa cells compared to T cells following exposure to mechanical deformation assisted RNP delivery could be due to the fact that HeLa cells have multiple copies of the B2M gene compared to other cell lines.

Mechanical deformation assisted co-delivery of Cascade Blue dextran, RNPs targeting the B2M gene (SEQ ID NOs. 1-4), and mRNA encoding a GFP expression construct was next assessed in human T cells. Unstimulated T cells were mixed with 100 μg/ml RNP targeting the B2M gene (SEQ ID NOs. 1-4), mRNA encoding the GFP expression construct, and 100 μg/ml 3 kDa Cascade Blue dextran (Thermo, Waltham, MA) at a final concentration of 20 M/ml in Optimem (Gibco). T cells were cultured and washed at 37° C. for 48 hours in complete T cell media following mechanical deformation before conducting genome editing and mRNA delivery efficiency analyses via flow cytometry. As shown in FIG. 3G, control cells not exposed to mechanical deformation displayed high levels of B2M expression and low levels of GFP fluorescence (Quadrant 1, 97.1% of cells). In contrast, T cells which were exposed to mechanical deformation forces showed low levels of B2M expression and high levels of GFP expression (Quadrant 3, 88.9% of cells) (FIG. 3G). Further flow cytometry-based efficiency analysis in control and mechanically deformed cells showed that 0% of control cells and 85.7% of mechanically deformed cells expressed GFP, successfully edited the B2M gene, and were cascade blue positive (FIG. 3H). These results show that the mechanical deformation assisted delivery techniques described herein can deliver chemically diverse cargos with surprisingly high efficiency in T cells.

Co-delivery of two distinct genome editing complexes in T cells was next assessed. Unstimulated T cells were mixed with 100 μg/ml RNP targeting the B2M gene (SEQ ID NO. 2), and 100 μg/ml RNP targeting the T-cell receptor (TCR) a constant (TRAC) (SEQ ID NO. 7) gene at a final concentration of 20 M/ml in Optimem (Gibco). T cells were cultured and washed at 37° C. for 48 hours in complete T cell media following mechanical deformation before conducting cell viability and genome editing efficiency analyses via flow cytometry. A cell viability analysis was conducted following mechanical deformation assisted genome editing by staining cells with the Live/Dead Near-infrared (Thermo) stain and quantified using flow cytometry. As shown in FIG. 3I, cell viability was only modestly affected following mechanical deformation assisted genome editing. Expression of the B2M and TRAC genes were assessed by immunolabelling T cells with an anti-human β2-microglobulin (β2M) antibody (BioLegend, 395712) and an anti-human TRAC antibody (BioLegend, 306716) and measuring fluorescence by flow cytometry. As shown in FIG. 3J, 59.2% of T cells exposed to mechanical deformation forces successfully edited both the B2M and TRAC genes compared to 0% of untreated control cells. Furthermore, as shown in FIG. 3K, few mechanically deformed T cells displayed high levels of expression of both B2M and TRAC (Quadrant 2, 8.62% of cells). In mechanically deformed cells, the single knockout rate for the TRAC gene was 90%, while the single knockout rate for the B2M gene was 60%. The difference in single knockout rate may be due to differences in guide RNA efficiencies.

These results demonstrate that successful editing of multiple genes with different guides via simultaneous delivery is possible using mechanical deformation assisted genome editing. These results thus represent a significant advance in genome editing methods and allow for an unexpectedly high correlation of knockout efficiency between the multiple targets, indicating that target cells received all cargos present during the mechanical deformation. In contrast, other commercially available genome editing techniques utilizing viral or nanoparticle based delivery methods only allow for stochastic delivery of cargo, resulting in limited efficacy in co-delivering multiple cargos to a target cell simultaneously.

### Example 4: Sequential Genome Editing and Complex Delivery in T Cells

This example demonstrates the ability of mechanical deformation to successfully deliver genome editing complexes and nucleic acids sequentially to target cells without severely impacting cell viability.

Sequential delivery of genome editing complexes and/or nucleic acid expression vectors to target cells while maintaining high delivery efficiency and cell viability represents a significant limitation of current transfection and transformation approaches. Current approaches utilizing viral, physical non-viral, and chemical non-viral approaches often require significant rest periods between genome editing steps to ensure cell viability remains at an acceptable level. In addition, each step in the cloning process must generally be performed and validated before advancing to a subsequent cloning steps.

Sequential delivery of target material to T cells was next assessed using mechanical deformation in T cells. Human T cells were obtained from frozen PBMCs and prepared for mechanical deformation assisted genome editing as disclosed in Example 1. As a first editing step, T cells were mixed with 100 μg/ml RNP targeting the TRAC gene (SEQ ID NO. 5-8) gene at a final concentration of 20 M/ml in Optimem (Gibco). The T cells were then subjected to first mechanical deformation step by passing the cells through low-porosity microsieves (Aquamarijn) to deliver the RNPs targeting the TRAC gene. After activation and proliferation for 5 days, the T cells were mixed with 100 μg/ml RNP targeting the B2M gene (SEQ ID NOs. 1-4) and mRNA encoding a GFP expression construct (SEQ ID NO. 9) at a final concentration of 20 M/ml in Optimem (Gibco). The T cells were then subjected to second mechanical deformation step to deliver the RNPs targeting the B2M gene and the GFP expression construct. The T cells were cultured and washed at 37° C. for 48 hours in complete T cell media following the two mechanical deformation steps before conducting cell viability and genome editing efficiency analyses via flow cytometry.

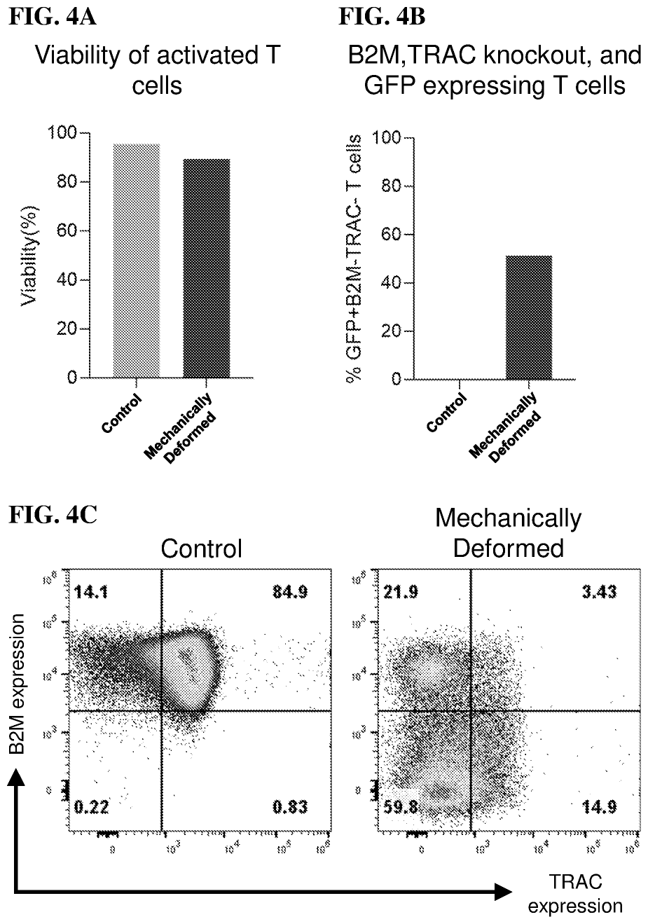

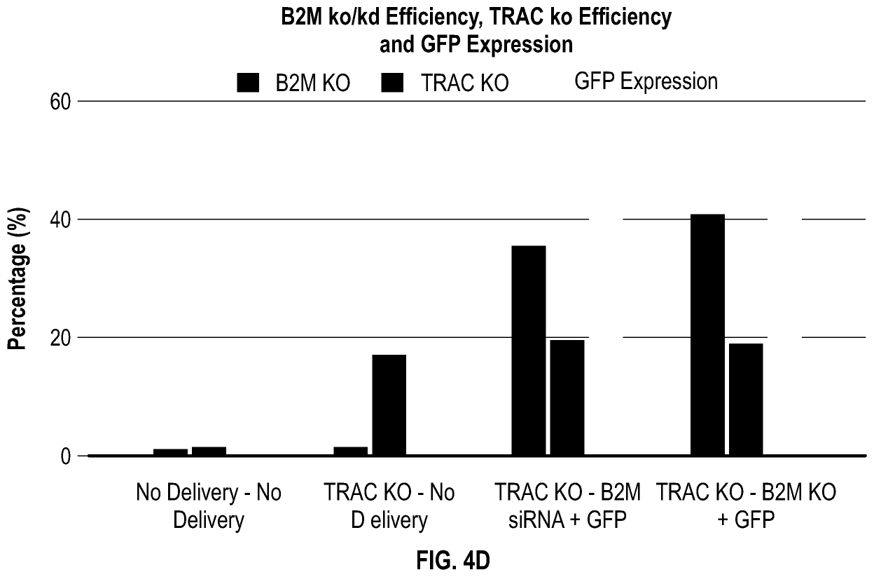

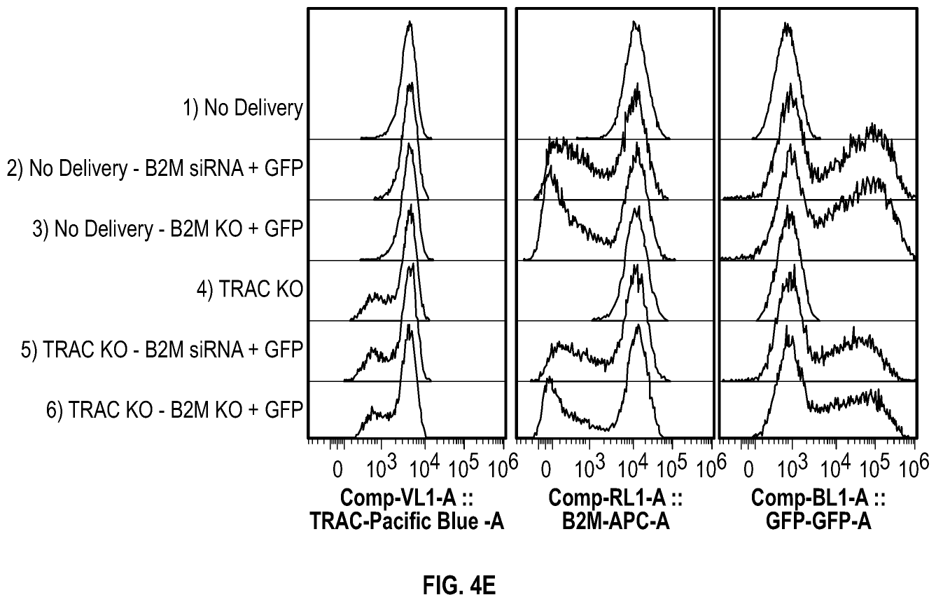

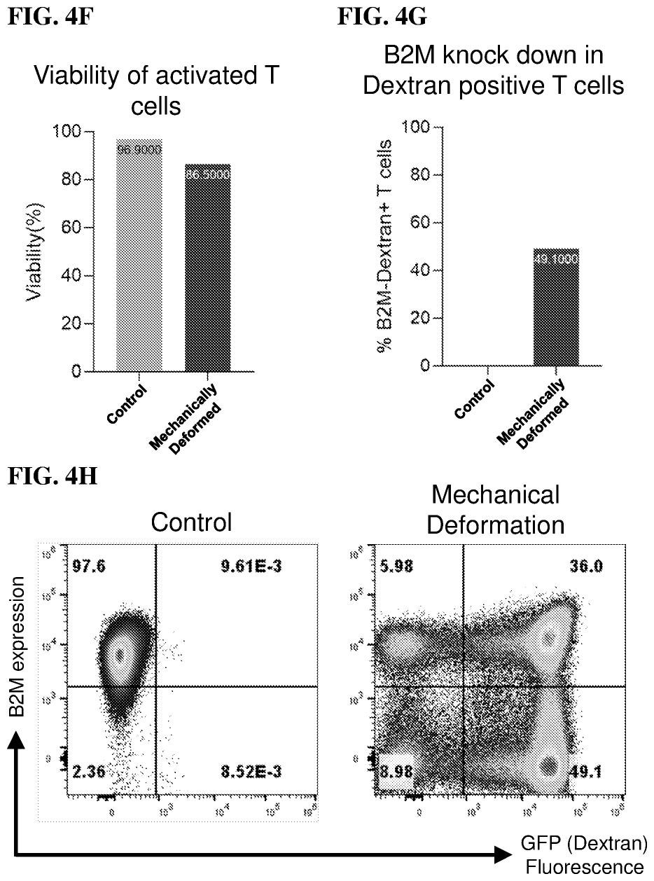

A cell viability analysis was conducted following mechanical deformation assisted genome editing by staining cells with the Live/Dead Near-infrared (Thermo) stain and quantified using flow cytometry. As shown in FIG. 4A, cell viability was only modestly affected following a cloning approach utilizing two sequential mechanical deformation steps. Expression of the B2M and TRAC genes were assessed by immunolabelling T cells with an anti-human β2-microglobulin (β2M) antibody (BioLegend, 395712) and an anti-human TRAC antibody (BioLegend, 306716) and measuring fluorescence by flow cytometry. Successful delivery of the GFP expression construct was assessed by measuring GFP fluorescence directly. As shown in FIG. 4B, the successful delivery of two genome editing complexes and an mRNA expression construct in two successive mechanical deformation steps was achieved in about 51% of T cells. Control cells which did not undergo the mechanical deformation forces did not successfully edit the B2M or TRAC genes and did not express GFP (FIG. 4B). As shown in FIG. 4C, about 60% of mechanically deformed T cells expressed low levels of both TRAC and B2M, indicating a surprisingly effective double knockout rate for a successive cloning approach. In mechanically deformed cells, the single knockout rate for the TRAC gene was about 82%, while the single knockout rate for the B2M gene was about 75% (FIG. 4C).

Sequential delivery of multiple gene editing complexes in T cells was further assessed by combining the delivery of TRAC genome editing complexes with Dextran during a first delivery step followed by a sequential delivery of GFP mRNA, and either B2M genome editing complexes or B2M siRNA. Human T cells were obtained from frozen PBMCs and prepared for mechanical deformation assisted genome editing as disclosed in Example 1. As a first editing step, T cells were mixed with 100 μg/ml RNP targeting the TRAC gene (SEQ ID NO. 5-8) gene and 100 μg/ml 10 kDa Cascade Blue dextran (Thermo, Waltham, MA) conjugated to AlexaFluor 647 fluorophores at a final concentration of 20 M/ml in Optimem (Gibco). The T cells were then subjected to first mechanical deformation step on day 0 by passing the cells through low-porosity microsieves with a porosity of 5 μm (Aquamarijn) under 10 PSI of pressure to deliver the RNPs targeting the TRAC gene and the 10 kDa dextran. The T cells were then activated by culturing the T cells in the presence of CD3/CD28 Dynabeads for 72 hours. Flow cytometry analysis was conducted on day 0 following the first mechanical deformation step to determine the delivery efficiency of the RNPs targeting the TRAC gene and the 10 kDa dextran and the viability of the T cells. On day 5, the activated T cells were mixed with mRNA encoding a GFP expression construct (SEQ ID NO. X) and either 100 μg/ml RNP targeting the B2M gene (SEQ ID NOs. 1-4) or siRNAs targeting the B2M gene at a final concentration of 20 M/ml in Optimem (Gibco). A second mechanical deformation step was performed on day 5 by passing the cells through low-porosity microsieves with a porosity of 7 μm (Aquamarijn) under 11 PSI of pressure to deliver the B2M siRNA, B2M sgRNA, and GFP expression construct. After a brief recovery period of 2 days, the B2M knockout/knockdown efficiency and GFP expression was quantified by flow cytometry analysis (day 7). Each mechanical deformation step was performed with about 6,000,000 cells.

As shown in FIGS. 4D and 4E, control cells which were not subjected to mechanical deformation forces during the first or second mechanical deformation step failed to knockout/knockdown B2M, knockout TRAC, or express GFP. As an additional control, cells which experienced mechanical deformation forces during the first mechanical deformation step but did not experience mechanical deformation forces during the second mechanical deformation step were able to knockout the TRAC gene, but failed to knockout/knockdown B2M or express GFP (FIG. 4D). Cells which experienced mechanical deformation forces during the first and second mechanical deformation steps successfully knocked out or knocked down B2M, edited the TRAC gene, and expressed GFP (FIG. 4D, FIG. 4E). These results demonstrate that applying mechanical deformation forces can greatly improve the delivery of diverse cargos to target cells using a variety of editing steps and approaches.

Sequential delivery of target material to T cells at clinical scale was assessed using mechanical deformation assisted genome editing. Human T cells were obtained from fresh leukopaks using whole blood pan T cell isolation kits (Miltenyi Biotec, 130-098-193). As a first editing step, T cells were mixed with 100 μg/ml RNP targeting the B2M gene (SEQ ID NOs. 1-4) gene at a final concentration of 40 M/ml in Optimem (Gibco). The T cells were then subjected to first mechanical deformation step by passing the cells through low-porosity chips (RM3) to deliver the RNPs targeting the B2M gene. T cells were then activated and expanded in G-Rex (Wilson Wolf) plates for 9 days. 220 million T cells were then mixed with 100 μg/ml 3 kDa Cascade Blue dextran (Thermo, Waltham, MA) at a final concentration of 40 M/ml in Optimem (Gibco). The T cells were then subjected to second mechanical deformation step to deliver the Cascade Blue dextran under 40 mL/min with an automated spinning membrane filtration system. Flow cytometry was then conducted to assess the viability and delivery of the cargo types to the T cells.

A cell viability analysis was conducted following mechanical deformation assisted genome editing by staining cells with the Live/Dead Near-infrared (Thermo) stain and quantified using flow cytometry. As shown in FIG. 4F, cell viability was only modestly affected following a genome editing approach utilizing two sequential mechanical deformation steps, with 96.9% of control T cells remaining viable and 86.5% of T cells subjected to mechanical deformation forces remaining viable. Expression of the B2M gene was assessed by immunolabelling T cells with an anti-human β2-microglobulin (β2M) antibody (BioLegend, 395712) and measuring fluorescence by flow cytometry. Successful delivery of Dextran was assessed by measuring GFP fluorescence directly. As shown in FIG. 4G, the successful editing of the B2M gene and successful delivery of Dextran was achieved in 49.1% of T cells subjected to mechanical deformation forces. Control cells which were not subjected to mechanical deformation forces did not successfully edit the B2M gene and did not deliver Dextran (FIG. 4G). As shown in FIG. 4H, about 85% of T cells subjected to mechanical deformation forces expressed high levels GFP fluorescence, indicating delivery of Dextran occurred at high efficiency. Additionally, about 58% of T cells subjected to mechanical deformation forces successfully edited the B2M gene (FIG. 4G).

These results provide evidence that mechanical deformation assisted genome editing represents a significant advance in the field of genome engineering. The methods described herein offer the potential for co-delivery of chemically distinct cargos with high editing efficiencies and with high cell viability. In addition, the methods described herein may be adapted for a sequential delivery approach without impacting cell viability or editing efficiency.

What is claimed is:

1. A method of delivering to a cell a plurality of different gene editing complexes targeting a gene or genetic motif, the method comprising:
subjecting a cell suspension to a mechanical deformation, the cell suspension comprising: (i) the cell, and (ii) the plurality of different gene editing complexes, or elements thereof,
wherein the mechanical deformation is configured such that at least two of the plurality of different gene editing complexes enters the cell.

2. The method of claim 1, wherein the mechanical deformation is configured such that at least one copy of each of the plurality of different gene editing complexes enters the cell.

3. The method of claim 1 or 2, wherein the at least one of the plurality of different gene editing complexes, or elements thereof, enters the cell as a complex.

4. The method of claim 1 or 2, wherein the least one of the plurality of different gene editing complexes enters the cell as two or more elements thereof.

5. The method of any one of claims 1-4, wherein the plurality of different gene editing complexes comprises two or more different gene editing complexes.

6. The method of any one of claims 1-5, wherein the plurality of different gene editing complexes comprises 2-100 different gene editing complexes.

7. The method of any one of claims 1-6, wherein the plurality of different gene editing complexes comprises a plurality of protein-nucleic acid complexes.

8. The method of claim 7, wherein the plurality of different gene editing complexes comprises a plurality of ribonucleoprotein (RNP) complexes each comprising a protein element and a guide nucleic acid element.

9. The method of claim 8, wherein the plurality of RNP complexes comprises two or more RNP complexes comprising the same protein element and different guide nucleic acid elements.

10. The method of claim 9, further comprising obtaining the plurality of RNP complexes, wherein the obtaining comprises loading the protein element with a guide nucleic acid element from a library of two or more different guide nucleic acid elements.

11. The method of any one of claims 1-10, wherein the plurality of different gene editing complexes comprises a Cas protein element.

12. The method of any one of claims 1-7, wherein the plurality of different gene editing complexes comprises a transposase.

13. The method of any one of claims 1-12, wherein at least two of the plurality of different gene editing complexes target a respective editing region, and wherein the respective editing regions are non-overlapping.

14. The method of any one of claims 1-13, wherein at least two of the plurality of different gene editing complexes target a respective editing region, and wherein the respective editing regions are overlapping.

15. The method of any one of claims 1-14, wherein the plurality of different gene editing complexes is configured to edit one or both alleles of the gene.

16. The method of any one of claims 1-15, wherein the plurality of different gene editing complexes is configured to affect an insertion, deletion, modification, and/or substitution.

17. The method of any one of claims 1-16, wherein editing performed by the plurality of different gene editing complexes results in a knock-out of a gene.

18. The method of any one of claims 1-16, wherein editing performed by the plurality of different gene editing complexes results in a knock-in of a gene.

19. The method of any one of claims 1-16, wherein editing performed by the plurality of different gene editing complexes results in an epigenetic modification.

20. The method of any one of claims 1-19, wherein the plurality of different gene editing complexes edits B2M, TCRα subunit constant (TRAC), BCL11A, CCR5, or PD-1.

21. The method of any one of claims 1-20, further comprising delivering another gene editing complex to edit a second gene or genetic motif.

22. The method of claim 21, wherein the gene or genetic motif is different than the second gene or genetic motif.

23. The method of any one of claims 1-22, further comprising co-delivering another cargo.

24. The method of claim 23, wherein the other cargo is a nucleic acid or a polypeptide, or a complex comprising a combination of a nucleic acid and a polypeptide.

25. The method of claim 24, wherein the co-delivered nucleic acid is configured for genome integration.

26. The method of claim 24, wherein the co-delivered nucleic acid is configured for protein expression.

27. The method of claim 24, wherein the co-delivered nucleic acid is configured for knock-out or knock-down of a gene.

28. The method of claim 24, wherein the co-delivered nucleic acid is a DNA, RNA, siRNA, miRNA, or oligonucleotide.

29. The method of any one of claims 24-28, wherein the co-delivered nucleic acid is configured for expression of any one of more of IL-2, IL-12, IL-15, IL-21, BCL-2, CXCR4, CCR7, TCF7, FOXO3, FOXp3, or a puromycin resistance gene.

30. The method of any one of claims 1-29, wherein the mechanical deformation comprises passing the cell suspension, or a portion thereof, through a material having pores therethrough, a microfluidic device comprising a plurality of constrictions or confinements, or a microneedle or microneedle array, or subjecting the cell suspension, or a portion thereof, to high shear forces with or without an electric field.

31. The method of claim 30, wherein the mechanical deformation comprises passing the cell suspension, or a portion thereof, through the material having pores therethrough.

32. The method of claim 31, wherein the material having pores therethrough is a filter.

33. The method of claim 31 or 32, wherein the material having pores therethrough comprises a silicon-based material.

34. The method of claim 31 or 32, wherein the material having pores therethrough comprises a polymer-based material.

35. The method of claim 34, wherein the polymer-based material is a track-etched filter.

36. The method of claim 31 or 32, wherein the material having pores therethrough comprises a metal-based material.

37. The method of any one of claims 31-36, wherein the material having pores therethrough comprises pores having an average size of about 0.1 μm to about 300 μm.

38. The method of any one of claims 31-37, wherein the cell suspension is passed through the material having pores therethrough at about 1 psi to about 20 psi.

39. The method of any one of claims 31-38, wherein the cell suspension is passed through the material having pores therethrough at a cell speed of about 0.1 mm/second to about 5 m/second.

40. The method of any one of claims 1-39, wherein the cell suspension has a volume of about 10 μL to about 5 L.

41. The method of any one of claims 1-40, wherein the cell suspension has a cell density of at least about 1,000 cells/mL.

42. The method of any one of claims 1-41, wherein the cell suspension comprises a buffer.

43. The method of any one of claims 1-42, wherein the cell suspension comprises a plurality of the cells, and wherein, following the subjecting the cell suspension to the mechanical deformation, at least about 25% of the plurality of the cells receives at least one of the plurality of different gene editing complexes.

44. The method of any one of claims 1-43, wherein the cell suspension comprises a plurality of the cells, and wherein, following the subjecting the cell suspension to the mechanical deformation, at least about 25% of the plurality of the cells are edited according to the design of at least one of the plurality of different gene editing complexes.

45. The method of any one of claims 1-44, wherein the cell suspension comprises a plurality of the cells, and wherein, following the subjecting the cell suspension to the mechanical deformation, at least about 25% of the plurality of the cell are viable.

46. The method of any one of claims 23-45, wherein the cell suspension comprises a plurality of the cells, and wherein, following the subjecting the cell suspension to the mechanical deformation, at least about 25% of the plurality of the cells that comprise at least one edit according to the design of at least one of the plurality of different gene editing complexes also contain the co-delivered nucleic acid.

47. The method of any one of claims 1-46, wherein the cell is a somatic cell, or a stem cell, or a derivative thereof.

48. The method of any one of claims 1-47, wherein the cell is a PBMC, or a derivative thereof.

49. The method of any one of claims 1-48, wherein the cell is an immune cell.

50. The method of any one of claims 1-49, wherein the cell is a T cell, NK cell, monocyte, B cell, or dendritic cell.

51. The method of any one of claims 1-47, wherein the cell is a stem cell.

52. The method of any one of claims 1-47 and 51, wherein the cell is an induced pluripotent stem cell.

53. The method of any one of claims 1-52, wherein the cell is obtained, or derived, from an individual.

54. The method of claim 53, wherein the individual is a human.

55. A method of delivering to a cell a plurality of different ribonucleoprotein (RNP) complexes targeting a gene or a genetic motif, the method comprising:
passing a cell suspension through a material having pores therethrough under a condition to mechanically deform the cell such that the plurality of different RNP complexes can enter the cell,
wherein the cell suspension comprises: (i) the cell, and (ii) the plurality of different RNP complexes, or elements thereof.

56. A method of delivering to a mixture of cells a plurality of different gene editing complexes targeting a gene or a genetic motif,
wherein the mixture of cells comprises two or more different cell types,
the method comprising:
subjecting a cell suspension to a mechanical deformation, the cell suspension comprising: (i) the mixture of cells, and (ii) the plurality of different gene editing complexes, or elements thereof,
wherein the mechanical deformation is configured such that at least two of the plurality of different gene editing complexes enter at least two different cell types of the mixture of cells.

57. The method of claim 56, wherein the mixture of cells comprises different cell types.

58. The method of claim 56 or 57, wherein the mixture of cells comprises PBMCs.

59. The method of claim 57 or 58, wherein the mechanical deformation is configured such that at least one copy of each of the plurality of different gene editing complexes enters the portion of the mixture of cells comprising two or more cell types.

60. A method of delivering to a cell a plurality of different gene editing complexes targeting a gene or a genetic motif, and another cargo, the method comprising:
passing a cell suspension through a material having pores therethrough under a condition to mechanically deform the cell such that the plurality of different gene editing complexes and the other cargo can enter the cell,
wherein the cell suspension comprises: (i) the cell, (ii) the plurality of different gene editing complexes, or elements thereof, and (iii) the other cargo.

61. A system comprising:
(a) a cell suspension comprising (i) a cell, and (ii) a plurality of different gene editing complexes; and
(b) a device for subjecting the cell suspension to a mechanical deformation,
wherein the device is configured such that subjecting the cell suspension to the mechanical deformation allows the plurality of different gene editing complexes to enter the cell.

62. A method of delivering to a cell a plurality of different gene editing complexes targeting a gene or genetic motif, and one or more nucleic acids, the method comprising:
subjecting a cell suspension to a mechanical deformation, the cell suspension comprising: (i) the cell, (ii) the plurality of different gene editing complexes, or elements thereof, and (iii) the one or more nucleic acids.

63. A method of delivering to a cell a plurality of different gene editing complexes targeting a gene or genetic motif, and one or more nucleic acids, the method comprising:
subjecting a first cell suspension to a mechanical deformation, the cell suspension comprising: (i) the cell, and (ii) the plurality of different gene editing complexes, or elements thereof; and
subjecting a second cell suspension to a mechanical deformation, the second cell suspension comprising: (i) the cell, and (ii) the one or more nucleic acids.

64. The method of claim 62, wherein there is at least 1 day between the subjecting the first cell suspension to the mechanical deformation and the subjecting the second cell suspension to the mechanical deformation.

65. The method of claim 64, wherein the at least 1 day between the subjecting the first cell suspension to the mechanical deformation and the subjecting the second cell suspension to the mechanical deformation allows for cell expansion.

66. The method of any one of claims 63-65, further comprising culturing the cell.

67. The method of any one of claims 62-66, wherein the one or more nucleic acids comprises one nucleic acid comprising RNA.
